(19) United States
(12) Patent Application Publication
(10) Pub. No.: US 2024/0201121 A1
(43) Pub. Date: Jun. 20, 2024

(54) ENZYME-LINKED IMMUNOSORBENT SENSOR ("ELIS-S")

(71) Applicant: PhageTech, Inc., Irvine, CA (US)

(72) Inventors: Jeffrey S. Briggs, Irvine, CA (US); Ahmon H. Brooks-Starks, Irvine, CA (US); Rodney Brenneman, Irvine, CA (US); Marcy Akhlaghpour, Irvine, CA (US); Tamara J. Elenberger, Irvine, CA (US)

(21) Appl. No.: 18/391,302

(22) Filed: Dec. 20, 2023

Related U.S. Application Data
(60) Provisional application No. 63/476,332, filed on Dec. 20, 2022.

(51) Int. Cl. G01N 27/327; G01N 27/416; G01N 33/543; G01N 33/574

(52) U.S. Cl. CPC G01N 27/3276; G01N 27/3271; G01N 27/3277; G01N 27/4161; G01N 33/5438; G01N 33/57407

(57) ABSTRACT

An enzyme-linked immunology-based sensors and biosensors, including systems and methods thereof are described herein. The novel use of electrochemical techniques in conjunction with enzyme-linked immunosorbent assay (ELISA) techniques to allow for the detection and/or quantification of a target in a sample, and thus allows for significant improvements over existing approaches, including decreased time required for testing a sample. The sensors described herein are particularly appropriate for applications requiring rapid results, for example, testing in the point of care setting in healthcare applications. Additionally, sensors described herein may also have particular usefulness in monitoring for the recurrence of bladder cancer.

### CROSS-REFERENCE TO RELATED APPLICATIONS

This application is a non-provisional and claims benefit of U.S. Provisional Application No. 63/476,332 filed Dec. 20, 2022, the specification of which is incorporated herein in their entirety by reference.

### FIELD OF THE INVENTION

The present invention relates to the field of sensors and biosensors, particularly enzyme-linked immunology-based sensors and biosensors. More specifically, the present invention relates to the novel use of electrochemical techniques in conjunction with enzyme-linked immunosorbent assays (ELISA) techniques to allow for the detection and/or quantification of a target in a sample.

### BACKGROUND OF THE INVENTION

Enzyme-linked immunosorbent assay (ELISA) is a form of enzyme immunoassay used to detect the presence of a ligand (analyte) in a liquid sample. ELISA utilizes antibodies specific to the ligand of interest. ELISA has a wide variety of applications in medicine, biotechnology, and other industries. In brief, the antigen of interest from the sample is adhered to a surface, either via adsorption (which is non-specific) or via capture by another antibody specific to the antigen, in the case of “sandwich” ELISA (which is specific). Detection antibodies, which are linked to an enzyme are then added, which form a complex with the antigen. Between steps, the plate is usually washed to remove any unbound or non-specifically bound proteins and/or antibodies. After final washing, an enzymatic substrate is introduced, which produces a signal, indicating the quantity of antigen in the sample. Traditionally, this signal has been a visible signal, typically a change in color. Generally, the greater the degree of color change, the higher the concentration of antigen in the sample. A spectrometer may optionally be used to provide quantitative data.

Medical diagnostics are increasingly being decentralized from hospital settings and into primary care, specialty clinics, and home care. ‘Point-Of-Care’ (POC) or ‘Point-Of-Need’ (PON) diagnostic products have been recognized as particularly valuable for cancer diagnosis or therapy monitoring. POC diagnostics have the potential to lower costs, provide earlier recognition of disease, and are more convenient for patients and physicians, which can improve adherence to monitoring protocols. POC diagnostics may work by identifying abnormal levels of recognized biomarkers in the body fluids of patients. These biomarkers may include small molecules, carbohydrates, lipids, proteins, nucleic acids, or any other compounds that have been found to be associated with certain diseases, including but not limited to cancer. Bladder cancer is a particularly attractive target for an effective POC test since it requires regular life-long surveillance due to its high recurrence rate.

POC diagnostic products in the area of urine sampling have encountered challenges as urine is a ‘waste stream’ for the body and contains uncontrolled levels of many different chemicals, as well as residual medications. Urine can thus have matrix effects and ‘foul’ typical sensors unless it is filtered, centrifuged, diluted, or otherwise pre-treated, which is usually not practical in the POC setting. Previous POC diagnostic products, especially those for bladder cancer, have been unable to overcome this technical challenge. Improved POC diagnostics, including but not limited to those for bladder cancer, have the potential to lower cost, provide earlier recognition of disease, and increase convenience for patients and physicians, which can, among other benefits, improve adherence to monitoring protocols. It is thus an objective of the present invention to provide improved POC diagnostics for purposes including but not limited to the surveillance of bladder cancer.

### BRIEF SUMMARY OF THE INVENTION

It is an objective of the present invention to provide systems and methods that allow for the electrochemical analysis of a sample (e.g., a urine sample), wherein a target market potentially present in a sample may be detected and/or quantified, as specified in the independent claims. Embodiments of the invention are given in the dependent claims. Embodiments of the present invention can be freely combined with each other if they are not mutually exclusive.

In some embodiments, the present invention comprises an enzyme-linked immunosorbent sensor. In other embodiments, the present invention comprises an enzyme-linked immunosorbent biosensor (ELIS-B). As used herein, the term “ELIS-B” is used to describe the present invention both as a sensor and as a biosensor. In some embodiments, the ELIS-B is a Faradaic electrochemical based test device and/or system with the potential to achieve sensitivity levels approaching those of widely accepted lab-based Enzyme-Linked Immunosorbent Assay (ELISA) tests. In some embodiments, the present invention involves the step of attaching a capture molecule or capture antibody to a working electrode surface. In some embodiments, the present invention involves the step of blocking the remaining surface of the working electrode to prevent unwanted signals. In some embodiments, the remaining surface of a counter electrode and/or a pseudo-reference electrode is also blocked.

One of the unique and inventive technical features of the present invention is the combination of ELISA techniques with electrochemical techniques for the detection and/or quantification of analytes (e.g., amyloid-B polymers, leptin, inflammatory markers such interleukins and C-reactive protein) in a sample. To the best of the Inventor's knowledge, ELISA techniques have heretofore not been combined with electrochemical techniques such that an ELISA-based sensor system formed by the combination of ELISA systems and techniques with redox-based electrochemical techniques, for example, chronoamperometry, for the qualitative and/or quantitative detection of analytes. Without wishing to limit the invention to any theory or mechanism, it is believed that the technical feature of the present invention advantageously provides for sensor systems incorporating the present invention to be miniaturized and to perform sample analysis at much improved speeds. For example, using traditional ELISA techniques with, e.g., spectrophotometry, analysis of a sample can take anywhere from 90 minutes to five hours and requires the use of a substantially larger device than required by the present invention. Using the systems and methods of the present invention, the same analysis can be performed in less than 30 minutes and may be done on a handheld device that is roughly the size of a handheld digital glucose monitor. Furthermore, the operation of the device comprising the present invention is simpler and requires less technical expertise than performing prior ELISA techniques with, e.g., spectrophotometry. The faster speed of sample analysis, smaller size, and simpler operation of a device comprising the present invention, as compared to prior ELISA-based approaches, makes the present invention particularly useful in a point-of-care setting. In addition to expediting sample processing, the present invention enhances sensitivity and specificity, resulting in markedly reduced error rates. None of the presently known prior references or work has the unique, inventive technical features of the present invention.

Furthermore, the inventive technical features of the present invention contributed to a surprising result. For example, ELISA traditionally incorporates the addition of a tether to specifically attach capture antibodies to a surface. While tethered antibodies increase attachment to surfaces during testing, the use of tethered antibodies is time-consuming, resource intensive, requires the use of specific solvents, affects the timing of analysis, and involves a different and more complicated procedure than the methods and systems of the present invention. The methods and systems of the present invention may optionally be used without the use of an antibody tether or capture molecule tether. Instead, by using passive adsorption, the present invention reduces the complexity of the testing process. Protein and buffer may be added directly over a surface, and molecules may be allowed to attach to the testing surface, for example, the surface of a gold working electrode. This avoids the use of a complex tether or particular solvents like DMSO. The testing process is simplified: one only needs to add protein and a buffer, as no solvent other than water is required.

This is a non-obvious and surprising result. One of ordinary skill in the art would expect that antibody tethers or capture molecule tethers would be required because the orientation of molecules, especially antibodies, must be specific with respect to the surface they are bound to in order for those antibodies or molecules to perform their capture function and bind a ligand. For example, the variable region of an antibody, forming part of the forked Y-shaped end of an antibody, is responsible for interacting with the target and grants the antibody its specificity to the target ligand. If the orientation of such an antibody were such that the portion of the antibody comprising the variable region was bound to the testing surface, the variable region would be unavailable to interact and bind with ligand, and thus the antibody would not work for ELISA. The use of a tether is expected to increase efficiency by orienting the capture molecule or capture antibody correctly, such that, for example, the variable region of an antibody is oriented away from the testing surface. Thus, one of ordinary skill in the art would expect that the lack of tether would result in an inadequate number of correctly oriented antibodies, such that ELISA or ELISA-like techniques performed without an antibody would fail. However, the present invention surprisingly produces accurate qualitative and quantitative results without the use of a capture antibody tether or a capture molecule tether. This represents a surprising and non-obvious result.

Moreover, for the same reasons stated above, the prior references teach away from the present invention. For example, because one of ordinary skill in the art would expect that a tether was necessary to correctly orient the capture antibodies or capture molecules, one of ordinary skill in the art would expect that systems or methods according to the present invention that did not incorporate a capture antibody tether or capture molecule tether would fail to produce a diagnostic signal of adequate quality. Thus, one of ordinary skill in the art would not attempt to utilize the present invention with a capture antibody tether or a capture molecule tether. Surprisingly, however, the use of the present invention without a capture antibody tether or a capture molecule tether is possible.

The present invention may feature a sensor system for analysis of a sample. The sensor system may comprise an electrode analysis device (e.g., a potentiostat), a working electrode comprising a surface, wherein capture molecules are adsorbed onto the surface of the working electrode to generate an occupied capture surface and a plurality of blocking molecules are adsorbed onto a remaining unoccupied capture surface; a counter electrode, a pseudo-reference electrode. In some embodiments, the surface of the working electrode is kept moist. The working electrode, the counter electrode, the pseudo-reference electrode, and the electrode analysis device are electrically connected. Additionally, the electrode analysis device analyzes at least one parameter selected from current or voltage in or between at least one of the working electrode, the counter electrode, or the pseudo-reference electrode.

The present invention may also feature a method of fabricating a sensor as described herein. The method comprises applying a capture antibody dissolved in a buffered solution to a surface of a working electrode, such that the capture antibody adsorbs onto the surface to generate an occupied capture surface. In some embodiments, hydration is maintained during this step such that the surface of the working electrode is kept moist. The method further comprises applying blocking molecules dissolved in a buffered solution to the surface of a sensor. The surface of the sensor may comprise all sample contacting surfaces, including all electrode surfaces, non-electrode surfaces, or a combination thereof. The blocking molecules may be adsorbed onto at least one of the electrode surfaces over a remaining unoccupied capture surface. Next, the buffered solution containing the blocking molecules is removed from the surface of the sensor. In some embodiments, the method further comprises placing a volumetric cell on the surface of the sensor. In some embodiments, the method further comprises washing the surface of the sensor with a buffered detergent.

The present invention may further feature a method of analyzing a sample using an electrochemical based sensor system. The method may comprise applying a sample possibly containing a target biomarker to the surface of an electrochemical based sensor as described herein. For example, the electrochemical based sensor may comprise an electrode analysis device (e.g., a potentiostat), a working electrode comprising a surface, wherein capture molecules are adsorbed onto the surface of the working electrode to generate an occupied capture surface and a plurality of blocking molecules are adsorbed onto a remaining unoccupied capture surface; a counter electrode, a pseudo-reference electrode. The working electrode, the counter electrode, the pseudo-reference electrode, and the electrode analysis device may be electrically connected. The method may further comprise applying a detection molecule and a redox-capable enzyme to the surface of the sensor and incubating the sample, the detection molecule, and the redox-capable enzyme on the sensor for a period of time, such that the target biomarker possibly present in the sample may bind the detection molecule to form the detection molecule-target biomarker complex. In some embodiments, the detection molecule is designed to bind to the target biomarker to form a detection molecule-target biomarker complex. Additionally, the capture molecule and the detection molecule-target biomarker complex form a sandwich complex upon binding of the capture molecule to the detection molecule-target biomarker complex, said sandwich complex comprising the capture molecules bound to the detection molecule-target biomarker complex. Lastly, the method comprises applying a substrate solution comprising a chromogenic substrate to the surface of the sensor. The redox-capable enzyme catalyzes a redox reaction in which the chromogenic substrate donates electrons to catalyze hydrogen peroxide into water; wherein the chromogenic substrate is thus oxidized to form an oxidized form of the chromogenic substrate; wherein the oxidized form of the chromogenic substrate comprises an electrically-detectable compound, wherein the electrically-detectable compound is reduced to an unoxidized form of the chromogenic substrate via a working electrode reducing potential applied to the working electrode, said working electrode reducing potential transfers electrons from the working electrode to the electrically-detectable compound, thus reducing the electrically-detectable compound to the unoxidized form of the chromogenic substrate, wherein a possible working electrode-counter electrode current change is thus generated, wherein said working electrode-counter electrode current change is measured as a function of time by a potentiostat using chronoamperometry, and wherein said working electrode-counter electrode current change may be used to perform at least one of detection of the target biomarker or quantification of the target biomarker.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows interactions occurring during fabrication of a sensor system of the present invention and during testing of a sample using a sensor system of the present invention. Capture molecules may be passively adsorbed onto the working electrode's capture surface, and a remaining unoccupied capture surface may be blocked with a plurality of blocking molecules. The target marker may bind to the detection molecule and to the capture molecule, forming the sandwich complex, and the probe-enzyme conjugate and/or redox-capable enzyme may catalyze a redox reaction with the redox reagent. The working electrode may be washed. The redox reagent may be applied, and a measurement may be recorded. Total testing time is typically less than 30 minutes, often around 28 minutes.

FIG. 2 shows an exemplary portion of a sensor system of the present invention. Visible are a working electrode, counter electrode, pseudo-reference electrode, and a volumetric cell on a single printed chip.

FIG. 3 shows an exemplary potentiostat/electrode analysis device of the present invention.

FIG. 4A and 4B show the effects of reducing the blocking step (which includes applying a plurality of bovine serum albumin molecules/ blocking molecules dissolved in a solution to surfaces of at least one of the working electrode(s), counter electrode, or pseudo-reference electrode) from two hours to 15 minutes (FIG. 4A) or 30 minutes (FIG. 4B). In general, this reduction in time did not substantially impact electrochemical tests, but non-specific binding was observed. FIG. 4A shows the change in ELISA charge versus bladder tumor antigen (BTA) concentration. Urine was diluted to 30% of the final volume for testing. N per group=3. Error bars=95% confidence interval of the means. Relative standard deviations of the means (CoV) are reported. Unpaired t-test with Welch's correction p=0.0020. FIG. 4B shows the change in ELISA charge versus bladder tumor antigen (BTA) concentration. Urine was diluted to 30% of the final volume for testing. N per group=3. Error bars=95% confidence interval of the means. Relative standard deviations of the means (CoV) are reported. Unpaired t-test with Welch's correction p=0.0052.

FIG. 5 shows a comparison of electrochemical results as a function of BTA concentration using an unpaired t-test with Welch's correction. The effects of washing the sensor only once, versus a greater number of times are demonstrated. If the sensor is washed once during sample testing, a difference in ELISA charge between BTA concentrations of 125 ng/ml and 0 ng/ml is still detectable, but the background signal increases when only a single wash is performed (indicated by a relatively high ELISA charge value where BTA concentration is zero). Urine was diluted to 30% final volume for testing. N per group=3. Error bars=95% confidence interval of the means. Relative standard deviations of the means (CoV) are reported. Unpaired t-test with Welch's correction p=0.0028. A follow-up experiment comparing groups head-to-head with different numbers of washes (three vs. one) might be performed.

FIG. 6A and FIG. 6B show the effects of further diluting samples, in this case urine samples, on the production of electrochemical results. Testing evaluated the recovery of 125 ng/ml BTA spiked into normal human urine (30 and 10% final concentration v/v). Further dilution of urine samples from 1/3 to 1/10 eliminates all apparent urinary matrix effects and enhances signals. FIG. 6A shows a comparison of electrochemical results with an anomalous chip in the 0% urine group. FIG. 6B shows a comparison of electrochemical results without the anomalous chip. N per group=3. Error bars=95% confidence interval of the means. Relative standard deviations of the means (CoV) are reported. Brown-Forsythe and Welch ANOVA test: P: 0.0184. P (outlier excluded): 0.0047. Dunnett's T3 multiple comparisons test: 30 vs. 10 Adjusted P: 0.0175. 30 vs. 0 Adjusted P: 0.4963. Outlier excluded: 0.0322. 10 vs. 0 Adjusted P: 0.8667. Outlier excluded: 0.8860.

FIG. 7A and FIG. 7B shows the effects of a six-point calibration using six different non-zero concentrations of BTA to evaluate a dose-response effect. FIG. 7A shows a dose-response curve for the baseline (zero)-corrected six non-zero calibrators using a two-hour block and five washes following the capture step. FIG. 7B shows the total error combining imprecision and inaccuracy using a two-hour block and five washes following the capture step. A follow-up calibration might be performed to better control for standard/testing variability and error.

FIG. 8A and FIG. 8B shows the effects of a repeated six-point calibration using six different non-zero concentrations of BTA to evaluate a dose-response effect and attempt to mitigate standard variability and error. FIG. 8A shows a dose-response curve for the baseline (zero)-corrected six non-zero calibrators using a two-hour block and five washes following the capture step. FIG. 7B shows the total error combining imprecision and inaccuracy using a two-hour block and five washes following the capture step. Future calibrations might include additional calibrators above 250 ng/ml and below 1.03 ng/ml to better anchor the model toward confirming the LLOQ, identifying the upper limit of quantitation (ULoQ), and improving upon fitting a curve to the data.

FIG. 9 shows an alternative embodiment of the sensor of the present invention. In this embodiment, a probe is omitted.

FIG. 10 shows an alternative embodiment of the sensor of the present invention. In this embodiment, the sensor features the use of a capture molecule-directed molecule. The capture molecule-directed molecule selectively binds the capture molecule. The capture molecule-directed molecule may be adsorbed onto the surface of the working electrode.

FIG. 11 shows an alternative embodiment of the sensor of the present invention. In this embodiment, the sensor features the use of streptavidin and/or avidin, which may be adsorbed onto the occupied capture surface of the working electrode. The capture molecule may be biotinylated, which may assist it in binding to the streptavidin and/or avidin.

### DETAILED DESCRIPTION OF THE INVENTION

Following is a list of elements corresponding to a particular element referred to herein:

050 Sensor

099 Potentiostat/Electrode analysis device

100 Working electrode

101 Gold surface/Working electrode surface

102 Capture antibody/Capture molecule/Biotinylated capture molecule

105 Blocking molecules/Bovine serum albumin molecules/Casein

200 Gold counter electrode/Counter electrode

300 Ag/AgCl pseudo-reference electrode/Pseudo-reference electrode

600 Sample

601 Target biomarker/Target marker

700 Detection molecule/Detection antibody

702 Detection molecule-target marker complex

703 Sandwich complex

750 Probe-enzyme conjugate

751 Enzyme/Redox-capable enzyme

752 Probe/Streptavidin/Avidin

753 Redox-capable enzyme

754 Capture molecule-directed antibody/Capture molecule-directed molecule

800 Volumetric cell

### Terms

Disclosed are various peptides, solvents, solutions, carriers, and/or components to be used to prepare compositions to be used within the methods disclosed herein. Also disclosed are the various steps, elements, amounts, routes of administration, symptoms, and/or treatments that are used or observed when performing the disclosed methods, as well as the methods themselves. These and other materials, steps, and/or elements are disclosed herein, and it is understood that when combinations, subsets, interactions, groups, etc. of these materials are disclosed, that while specific reference of each various individual and collective combination and permutation of these compounds may not be explicitly disclosed, each is specifically contemplated and described herein. It is also to be understood that the terminology used herein is for the purpose of describing particular embodiments only and is not intended to be limiting.

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessary solely”. Furthermore, variation of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

Suitable methods and materials for the practice and/or testing of embodiments of the disclosure are described below. Such methods and materials are illustrative only and are not intended to be limiting. Other methods and materials similar or equivalent to those described herein can be used. For example , conventional methods well known in the art to which the disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999, Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.), the disclosures of which are incorporated in their entirety herein by reference.

All publications, patent applications, patents, and other references mentioned herein are incorporated by reference in their entirety for all purposes. In case of conflict, the present specification, including explanations of terms, will control.

Although methods and materials similar or equivalent to those described herein can be used to practice or test the disclosed technology, suitable methods and materials are described below. The materials, methods, and examples are illustrative only and not intended to be limiting.

As used herein, the terms “subject” and “patient” are used interchangeably. As used herein, a subject can be a mammal such as a non-primate (e.g., cows, pigs, horses, cats, dogs, rats, etc.) or a primate (e.g., monkey and human). In specific embodiments, the subject is a human. In one embodiment, the subject is a mammal (e.g., a human) having a disease, disorder or condition described herein. In another embodiment, the subject is a mammal (e.g., a human) at risk of developing a disease, disorder or condition described herein. In certain instances, the term patient refers to a human.

As used herein, a “biological sample” or “clinical sample” may be used interchangeably and may refer to any biological material taken from a subject. Non-limiting examples of a biological material to be taken for a biological sample may include but are not limited to, urine, saliva, or blood.

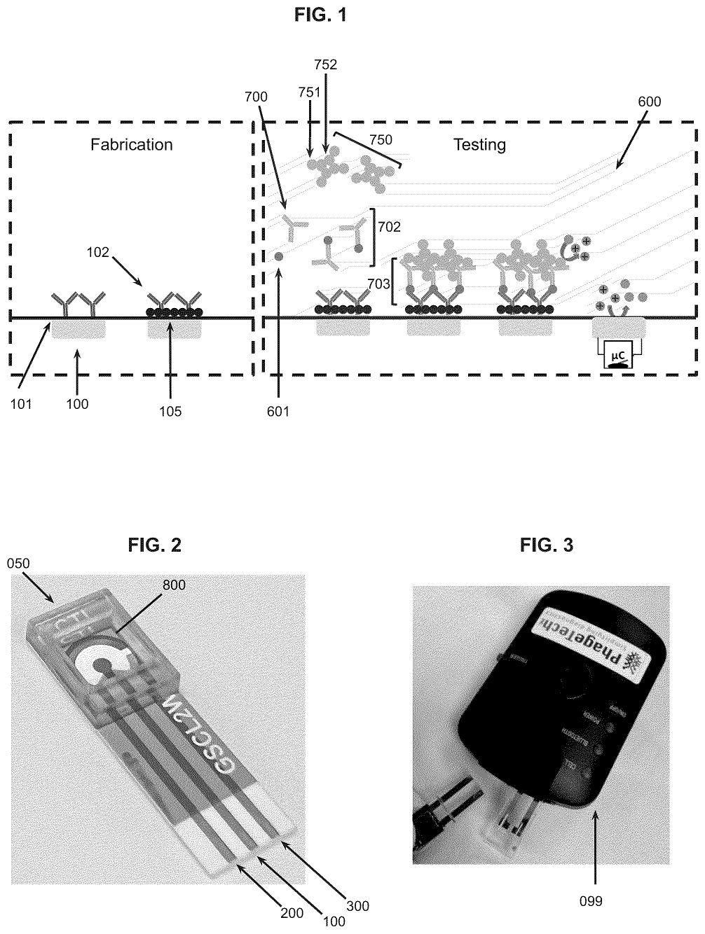

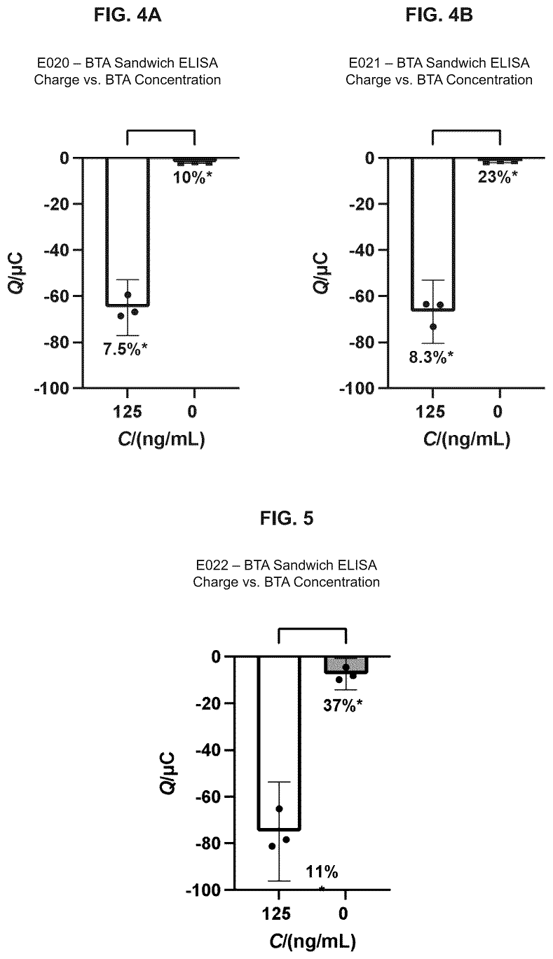

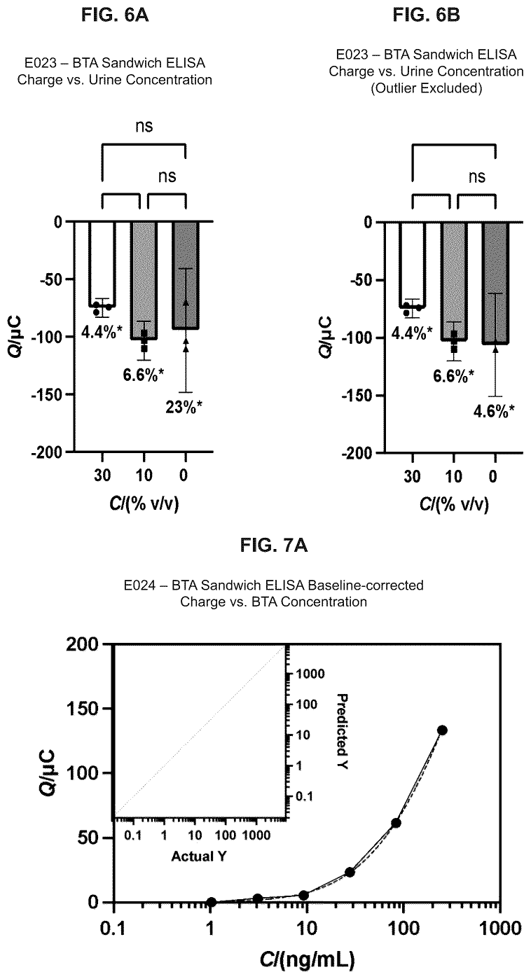

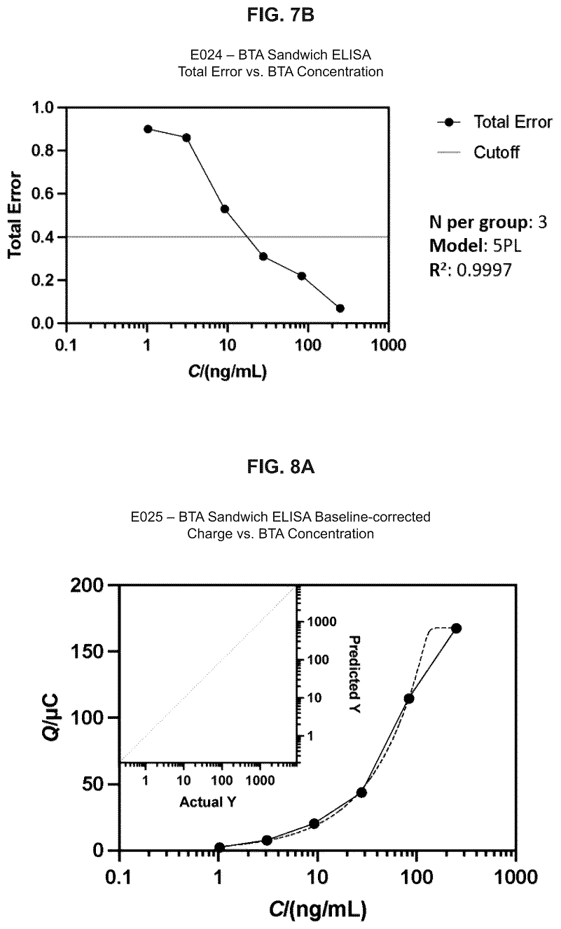

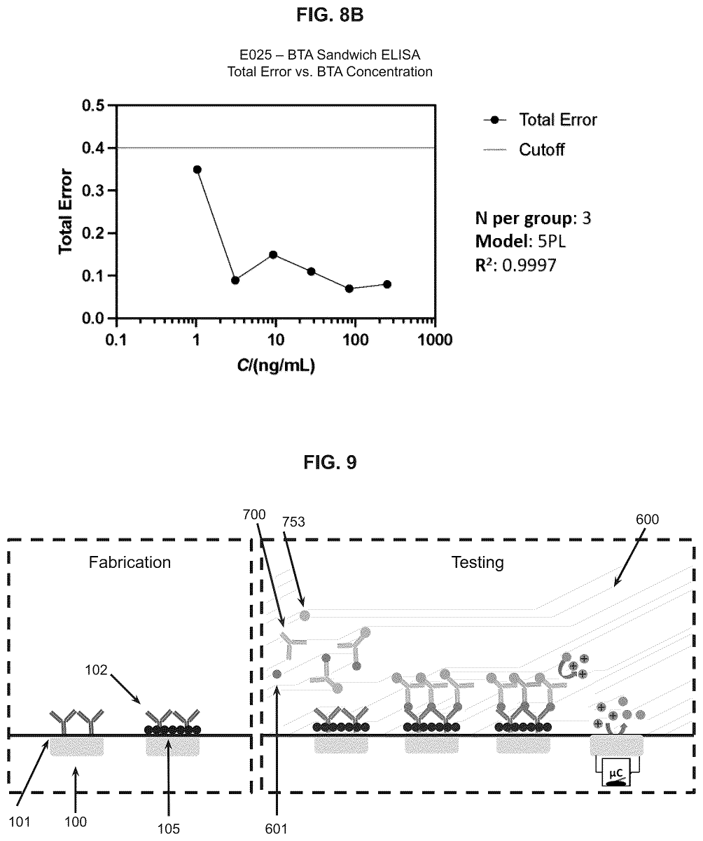

Referring now to FIGS. 1-11, the present invention features a sensor system for analysis of a sample.

### Sensor System

The present invention features a Faradaic electrochemical based sensor system (e.g., an Enzyme-Linked Immunosorbent Biosensor (ELIS-B). The sensor system may comprise a potentiostat (099), a working electrode (100) comprising a surface (101), and a capture molecule (102; e.g., a capture antibody) adsorbed (e.g., passively) onto the surface (101) to generate an occupied capture surface. The sensor system may further comprise blocking molecules (105; e.g., bovine serum albumin molecules or Casein molecules) disposed on the unoccupied captured surface.

In some embodiments, a remaining unoccupied capture surface is occupied by a plurality of blocking molecules (105). In other embodiments, a remaining unoccupied capture surface is occupied by a plurality of bovine serum albumin molecules. In other embodiments, a remaining unoccupied capture surface is occupied by a plurality of casein molecules (105). Non-limiting examples of blocking molecules (150) include bovine serum albumin (BSA) molecules, Casein molecules, or a combination thereof. Other blocking molecules may be used in accordance with the present invention. As used herein, “blocking molecules” may refer to molecules (e.g., a plurality of molecules) that decrease non-specific binding.

The aforementioned sensor system may further comprise a counter electrode (200) and a pseudo-reference electrode (300). In some embodiments, the working electrode (100), the counter electrode (200), the pseudo-reference electrode (300), and the potentiostat (099) are electrically connected.

In some embodiments, the sensor system comprises at least one working electrode (100), as described herein, a counter electrode (200), and a pseudo-reference electrode (300). In other embodiments, the sensor system comprises two working electrodes (100) as described herein, a counter electrode (200) and a pseudo-reference electrode (300). In some embodiments, the sensor system comprises three working electrodes (100) as described herein, a counter electrode (200), and a pseudo-reference electrode (300). In other embodiments, the sensor system comprises four working electrodes (100) as described herein, a counter electrode (200), and a pseudo-reference electrode (300). In further embodiments, the sensor system comprises up to ten working electrodes (100) as described herein, a counter electrode (200), and a pseudo-reference electrode (300).

In some embodiments, the working electrode (100) is a gold electrode. In other embodiments, the working electrode (100) is a carbon electrode. In further embodiments, the working electrode (100) is a silver electrode. In some embodiments, the counter electrode (200) is a gold electrode. In other embodiments, the counter electrode (200) is a carbon electrode. In further embodiments, the counter electrode (200) is a silver electrode. In some embodiments, the reference electrode (300) is a silver/silver chloride (Ag/AgCl) electrode. In certain embodiments, the system comprises a gold working electrode (100), a gold counter electrode (200), and an Ag/AgCl pseudo-reference electrode (300). In some embodiments, at least one of the working electrode (100), counter electrode (200), or pseudo-reference electrode (300) comprise carbon. In other embodiments, the system comprises a carbon working electrode (100), a carbon counter electrode (200), and an Ag/AgCl pseudo-reference electrode (300). In some embodiments, the system comprises a silver working electrode (100), a silver counter electrode (200), and an Ag/AgCl pseudo-reference electrode (300).

In some embodiments, the potentiostat (099) controls a voltage difference between the working electrode (100) and the Ag/AgCl pseudo-reference electrode (300) by injecting current through the counter electrode (200). In some embodiments, the potentiostat (099) measures a working electrode-counter electrode current change at the working electrode (100).

In some embodiments, the present invention further comprises a capture molecule-directed molecule, i.e., a molecule that binds specifically to the capture molecule. In some embodiments, the capture molecule-directed molecule is a capture molecule-directed antibody, i.e., an antibody that binds specifically to the capture molecule. In other embodiments, the capture molecule-directed molecule is a capture molecule-directed antigen, i.e., an antigen that binds specifically to the capture molecule. In further embodiments, the capture molecule-directed molecule is a capture molecule-directed protein, i.e., a protein that binds specifically to the capture molecule.

In some embodiments, the capture molecule is an antibody (e.g., a capture antibody). In other embodiments, the capture molecule is an antigen (e.g., a capture antigen). In further embodiments, the capture molecule is a protein (e.g., a capture protein). As used herein, a “capture molecule” refers to a molecule that binds to a detection molecule as described herein and/or a target biomarker or antigen.

The sensor system may further comprise a substrate, e.g., made of polyethylene terephthalate (PET). For example, the electrodes described herein (e.g., the working electrode, the counter electrode, and the reference electrode) may be ablated or plated on top of the substrate. A dielectric layer is then placed on top of any electrode surface area that is not intended to be exposed to the sample. In some embodiments, the substrate is made of ceramic (such as alumina or glass). In other embodiments, the substrate is made of other polymers. The substrates described herein are not limited to the aforementioned materials. In some embodiments, the sensor system further comprises an insulating dielectric (i.e., a dielectric layer). The insulating dielectric may be used to prevent exposure to certain parts of the electrodes described herein (e.g., the working electrode, the counter electrode, and the reference electrode).

In some embodiments, the sensor system may further comprise a biological sample (600), possibly containing a target biomarker (601) and a detection molecule (700; e.g., a detection antibody or a detection protein). In some embodiments, the detection molecule is an antibody (e.g., a detection antibody). In other embodiments, the detection molecule is a protein (e.g., a detection protein). Non-limiting examples of a detection molecule include but are not limited to peptides, proteins, hormones, or antibodies.

In some embodiments, the detection molecule (700; e.g., the detection antibody) is biotinylated (e.g., bound to a biotin molecule, e.g., at least one biotin molecule). In other embodiments, the detection molecule (700) is bound to a streptavidin or avidin molecule (e.g., at least one streptavidin or avidin molecule).

In other embodiments, the detection molecule (700; e.g., the detection antibody) is bound to an enzyme (e.g., a redox-capable enzyme). In some embodiments, the enzyme (e.g., a redox-capable enzyme) is horseradish peroxidase. In further embodiments, the detection molecule (700; e.g., the detection antibody) is bound to a protein (e.g., a green fluorescent protein (GFP)). In some embodiments, the detection molecule (700; e.g., the detection antibody) is bound to a GFP. In some embodiments, the detection molecule (700) is bound to streptavidin or avidin.

In some embodiments, the detection molecule (700; e.g., the detection antibody) is designed to bind to the target biomarker (601) to form a detection antibody-target biomarker complex (702). In some embodiments, the capture molecule (102; e.g., a capture antibody) and the detection antibody-target biomarker complex (702) form a sandwich complex (703) upon binding of the capture molecule (102; e.g., a capture antibody) to the detection antibody-target biomarker complex (702). In some embodiments, this binding event occurs via interactions between the capture antibody (102) and a target biomarker portion of the detection antibody-target biomarker complex (702). In some embodiments, the sandwich complex (703) comprises the capture molecule (102; e.g., a capture antibody) bound to the detection antibody-target biomarker complex (702).

In some embodiments, the sensor system further comprises a probe-enzyme conjugate (750). In some embodiments, the probe-enzyme conjugate (750) comprises a probe (752; e.g., a streptavidin or avidin tetramer) bound to an enzyme (751). In some embodiments, the enzyme comprises a redox-capable enzyme. In some embodiments, the redox-capable enzyme is horseradish peroxidase.

The probe-enzyme conjugate (750) is designed to interact with the sandwich complex (703). For example, the probe (752; e.g., a streptavidin or avidin tetramer) of the probe-enzyme conjugate (750) may interact with the biotin bound to the detection molecule (700; e.g., detection antibody) of the sandwich complex (703). In other embodiments, the horseradish peroxidase of the probe-enzyme conjugate (750) interacts with the biotin bound to the detection antibody (700) of the sandwich complex (703).

In some embodiments, the sensor system described herein may utilize a detection molecule (700; e.g., detection antibody) comprising both a target biomarker (601) and an enzyme (751; e.g., a redox-capable enzyme) bound thereto. For example, a detection antibody may comprise an enzyme (751; e.g., a redox-capable enzyme) bound to a variable region of the enzyme (e.g., at least a portion of a first variable region). A second variable region of the detection antibody may bind a target biomarker (601). In some embodiments, the detection antibody may comprise an enzyme (751; e.g., a redox-capable enzyme) bound to the constant region of the enzyme, and a variable region of the detection antibody may bind a target biomarker (601). In other embodiments, the detection antibody may comprise an enzyme (751; e.g., a redox-capable enzyme) bound to a variable region of the enzyme (e.g., a portion first variable region), and a target biomarker (601) may bind to the same variable region (e.g., an alternate portion of the first variable region).

In some embodiments, the enzyme (751; e.g., a redox-capable enzyme) bound to the detection antibody is horseradish peroxidase. In some embodiments, the detection antibody (700) bound to both a target biomarker (601) and an enzyme is used in place of a probe-enzyme conjugate (see FIG. 9).

In some embodiments, the sensor systems comprise a substrate solution. As used herein, a “substrate solution” may refer to a solution that starts the development process either by colorimetric readout or oxidation/reduction process. The substrate solution may comprise a chromogenic substrate. Non-limiting examples of chromogenic substrates include but are not limited to 3,3′,5,5′-tetramethylbenzidine (TMB), 2,2′-Azino-bis-(3-ethylbenzothiazoline-6-sulfonic acid) (ABTS), or 3,3′-Diaminobenzidine (DAB). The development process may refer to the time it takes to see a change in the color of the substrate solution. For example, as the enzyme undergoes oxidation, the substrate solution may begin to yield a blue color that may be detected at either 372 nm or 652 nm.

In some embodiments, the substrate solution comprises phosphate-citrate buffer solution, hydrogen peroxide, and 3,3′,5,5′-tetramethylbenzidine (TMB). In some embodiments, the substrate solution may further comprise o-phenylenediamine, dihydrochloride, or peroxidase substrate buffer. In other embodiments, the solution may comprise 2,2′-Azino-bis-(3-ethylbenzothiazoline-6-sulfonic acid) (ABTS), diammonium salt, or other appropriate compounds. In some embodiments, ABTS may be used in place of TMB.

Without wishing to limit the present invention to any theory or mechanism, it is believed that the redox-capable enzyme (e.g., the horseradish peroxidase enzyme) bound to the detection molecule (700; e.g., the detection antibody) catalyzes a redox reaction wherein the chromogenic substrate (e.g., TMB) donates electrons to catalyze hydrogen peroxide into water. The chromogenic substrate (e.g., TMB) is thus oxidized to form an oxidized form of the chromogenic substrate (e.g., TMB). In some embodiments, the oxidized form of the chromogenic substrate (e.g., TMB) comprises an electrically-detectable compound (e.g., ferrocyanide). In some embodiments, the electrically detectable compound (e.g., ferrocyanide) is reduced to an unoxidized form of the chromogenic substrate (e.g., TMB) via a working electrode reducing potential applied to the working electrode (100). The working electrode reducing potential transfers electrons from the working electrode (100) to the electrically-detectable compound (e.g., ferrocyanide), thus reducing the electrically-detectable compound (e.g., ferrocyanide) to the unoxidized form of the chromogenic substrate (e.g., TMB). This generates the possible working electrode-counter electrode current change. In some embodiments, the working electrode-counter electrode current change is measured as a function of time by the potentiostat using chronoamperometry. In some embodiments, the working electrode-counter electrode current change is measured using differential pulse voltammetry. In some embodiments, the working electrode-counter electrode current change may be used to perform at least one of detection of the target biomarker (601) or quantification of the target biomarker (601).

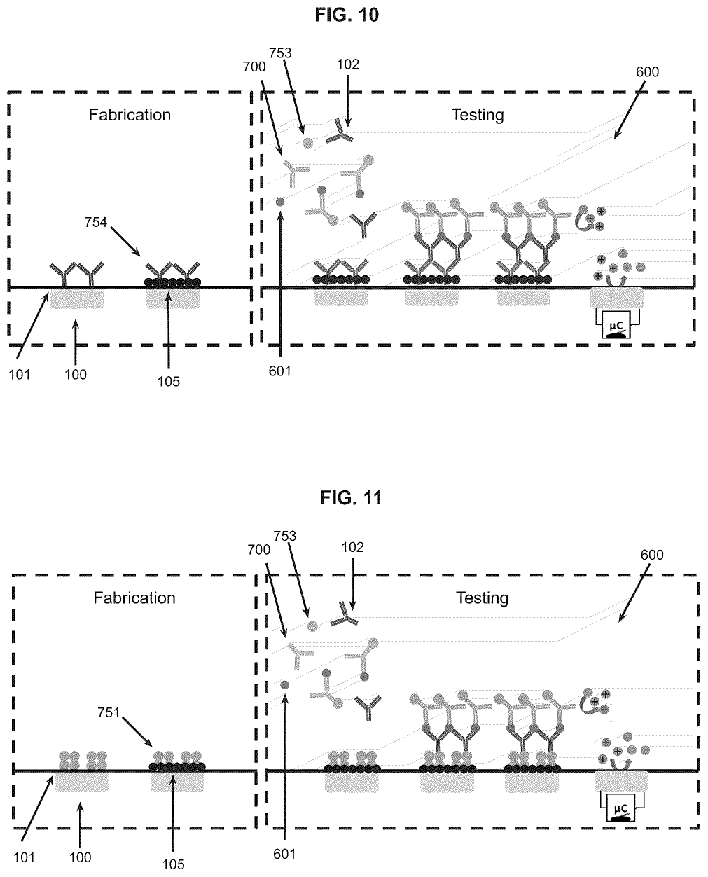

Referring to FIG. 10, the present invention may also feature a Faradaic electrochemical based sensor system (e.g., an Enzyme-Linked Immunosorbent Biosensor (ELIS-B) in which a capture molecule-directed molecule (754; e.g., a capture molecule-directed antibody), i.e., a molecule that binds specifically to the capture molecule bound to the surface (101) of the working electrode (100). In some embodiments, the capture molecule is a capture antibody. In some embodiments, the capture molecule-directed molecule (754) is a capture molecule-directed antibody.

In some embodiments, the sensor system comprises a potentiostat (099), a working electrode (100) comprising a surface (101), and a capture molecule-directed molecule (754) adsorbed (e.g., passively) onto the surface (101) to generate an occupied capture surface. The sensor may further comprise blocking molecules (105; bovine serum albumin molecules) disposed on the unoccupied captured surface.

In some embodiments, the capture molecule-directed molecule (754) is adsorbed onto the working electrode surface (101) to generate an occupied capture surface before the capture molecule (102), sample (600), and detection molecule (700) are added to the solution. In some embodiments, the capture molecule (102) is mixed in solution with the sample (600) and detection molecule (700) after the capture molecule-directed molecule (754) is added to the solution. In some embodiments, the capture molecule (102) is mixed in solution with the sample (600) and detection molecule (700) before the capture molecule-directed molecule (754) is added to the solution.

In some embodiments, the capture molecule (102) binds the target biomarker (601) to form a capture molecule-target marker complex. In some embodiments, the capture molecule-target marker complex is bound by the detection molecule to form a capture molecule-target marker-detection molecule complex. In some embodiments, the detection molecule is a detection antibody (700). In some embodiments, the capture molecule-target marker-detection molecule complex is bound by the capture molecule-directed molecule (754). In some embodiments, said capture molecule-directed molecule (754) is adsorbed onto the working electrode surface (101) over an occupied capture surface.

Referring to FIG. 11, the present invention may also feature a Faradaic electrochemical based sensor system (e.g., an Enzyme-Linked Immunosorbent Biosensor (ELIS-B) in which an avidin or streptavidin (752) tetramer is bound to the surface (101) of the working electrode (100).

In some embodiments, the sensor system comprises a potentiostat (099), a working electrode (100) comprising a surface (101), and an avidin or streptavidin (752) tetramer adsorbed (e.g., passively) onto the surface (101) to generate an occupied capture surface. The sensor may further comprise blocking molecules (105; bovine serum albumin molecules) disposed on the unoccupied captured surface.

In some embodiments, the capture molecule (102) is mixed in solution with the sample (600) and detection molecule (700). In some embodiments, the capture molecule (102) binds the target biomarker (601) to form a capture molecule-target marker complex. In some embodiments, the capture molecule-target marker complex is bound by the detection molecule to form a capture molecule-target marker-detection molecule complex.

In some embodiments, the detection molecule is a detection antibody (700). In some embodiments, the capture molecule (102) is biotinylated. In some embodiments, the biotinylated capture molecule (102) is bound by avidin and/or streptavidin.

Binding of avidin and/or streptavidin to the biotinylated capture molecule (102) thus allows avidin and/or streptavidin to bind the capture molecule-target marker-detection molecule complex. In some embodiments, avidin and/or streptavidin bind to the capture molecule (102) via the binding of avidin and/or streptavidin to one or more biotin molecules bound to the capture molecule (102).

In some embodiments, the avidin and/or streptavidin is/are adsorbed onto the working electrode surface (101) to generate an occupied capture surface. In some embodiments, the avidin and/or streptavidin is/are adsorbed onto the working electrode surface (101) to generate an occupied capture surface before blocking an unoccupied capture surface with a plurality of blocking molecules. In some embodiments, the avidin and/or streptavidin is/are adsorbed onto the working electrode surface (101) to generate an occupied capture surface before the capture molecule (102), sample (600), and detection molecule (700) are added to solution. In some embodiments, the plurality of blocking molecules comprises bovine serum albumin. In some embodiments, the capture molecule (102) is mixed in solution with the sample (600) and detection molecule (700) after the avidin and/or streptavidin is/are added to the solution. In some embodiments, the capture molecule (102) is mixed in solution with the sample (600) and detection molecule (700) before the avidin and/or streptavidin is/are added to the solution.

In some embodiments, the capture molecule (102) binds the target marker (601) to form a capture molecule-target marker complex. In some embodiments, the capture molecule-target marker complex is bound by the detection molecule to form a capture molecule-target marker-detection molecule complex. In some embodiments, the detection molecule is a detection antibody (700). In some embodiments, the capture molecule-target marker-detection molecule complex is bound by avidin and/or streptavidin. In some embodiments, said avidin and/or streptavidin is/are adsorbed onto the working electrode surface (101) over an occupied capture surface.

In some embodiments, the sensor system further comprises a vibration exciter to excite at least one of the capture antibodies (102), the plurality of bovine serum albumin molecules (105), the sample (600), the target biomarker (601), the detection antibody (700), the detection antibody-target biomarker complex (702), the sandwich complex (703), the probe-enzyme conjugate (750), the probe (751), the enzyme, the phosphate-citrate buffer solution, the hydrogen peroxide, or the TMB. In some embodiments, the vibration exciter is a vibration motor. In some embodiments, the vibration exciter is an electro-dynamic exciter. In some embodiments, the vibration exciter is a hydraulic exciter. In some embodiments, the vibration exciter is a mechanical exciter. In some embodiments, the vibration exciter is particularly useful to increase the dispersion of the capture antibody over the occupied capture surface and/or to increase the dispersion of the blocking antibody over the unoccupied capture surface.

The present invention features a sensor system for analysis of a sample. In some embodiments, the sensor system includes an electrode analysis device (099). In some embodiments, the electrode analysis device is a potentiostat. In other embodiments, the electrode analysis device is an amperostat, galvanostat, or other appropriate electrode analysis device. In some embodiments, the sensor system includes a working electrode (100). In some embodiments, the working electrode (100) includes a working electrode surface (101) and a capture molecule (102) adsorbed onto the working electrode surface over an occupied capture surface. In some embodiments, a remaining unoccupied capture surface is occupied by a plurality of blocking molecules (105). In some embodiments, the sensor system includes a counter electrode (200), and/or a pseudo-reference electrode (300). In some embodiments, the working electrode (100), counter electrode (200), pseudo-reference electrode (300), and electrode analysis device (099) are electrically connected. In some embodiments, the electrode analysis device (099) analyzes at least one parameter selected from current or voltage in or between at least one of the working electrode (100), counter electrode (200), or pseudo-reference electrode (300). In some embodiments, the sensor system includes a sample (600) possibly containing a target marker (601). In some embodiments, the sensor system includes a detection molecule (700). In some embodiments, the detection molecules (700) are designed to bind the target marker (601) to form a detection molecule-target marker complex (702). In some embodiments, the capture molecule (102) and the detection molecule-target marker complex (702) form a sandwich complex (703) upon binding of the capture molecule (102) to the detection molecule-target marker complex (702). In some embodiments, the sandwich complex (703) includes the capture molecule (102) bound to the detection molecule-target marker complex (702). In some embodiments, the sensor system includes a redox-capable enzyme (753). In some embodiments, the sensor system includes a redox reagent. In some embodiments, the redox-capable enzyme (753) performs at least one of electron donation or electron acceptance with the redox reagent, wherein the oxidation state of the redox reagent is changed to produce an oxidized or reduced redox reagent. In some embodiments, the oxidized or reduced redox reagent comprises an electrically-detectable compound. In some embodiments, the electrically-detectable compound is oxidized or reduced via a working electrode potential applied to the working electrode (100). In some embodiments, the working electrode potential transfers electrons to or from the working electrode (100) to or from the electrically-detectable compound, thus oxidizing or reducing the electrically-detectable compound to its original unoxidized or unreduced state. In some embodiments, a possible change in at least one of current or voltage in or between at least one of the working electrode (100), counter electrode (200), or pseudo-reference electrode (300) is thus generated. In some embodiments, the possible change in at least one of current or voltage in or between at least one of the working electrode (100), counter electrode (200), or pseudo-reference electrode (300) is measured by the electrode analysis device (099). In some embodiments, the potential possible change in at least one of current or voltage in or between at least one of the working electrode (100), counter electrode (200), or pseudo-reference electrode (300) may be used to perform at least one of detection of the target marker (601) or quantification of the target marker (601).

In some embodiments, the system further comprises a vibration exciter to excite at least one of the capture molecules (102), the plurality of blocking molecules (105), the sample (600), the target marker (601), the detection molecule (700), the detection molecule-target marker complex (702), the sandwich complex (703), the redox-capable enzyme (753), or the redox reagent.

In some embodiments, the working electrode surface (101) comprises carbon. In some embodiments, the working electrode surface (101) comprises gold.

### Methods of Fabrication

The present invention may also feature a method of fabricating a Faradaic electrochemical based sensor (e.g., an Enzyme-Linked Immunosorbent Biosensor (ELIS-B).

In some embodiments, the method includes placing a volumetric cell (800) onto the surface of a sensor (050). In some embodiments, the volumetric cell (800) is a flow cell. In some embodiments, the volumetric cell (800; e.g., the flow cell) is made of polylactic acid or polylactide (PLA). In some embodiments, the volumetric cell (800; e.g., the flow cell) comprises an opening that enables the addition of a sample (e.g., a urine sample, a blood sample, or a saliva sample) onto the sensor system as described herein.

As described above, the sensor system may comprise a working electrode (100) comprising a surface, a counter electrode (200), and a pseudo-reference electrode (300). In certain embodiments, the sensor systems comprise a gold working electrode (100) comprising a gold surface, a gold counter electrode (200), and an Ag/AgCl pseudo-reference electrode (300). The method may first comprise applying a capture molecule (102; e.g., capture antibody) dissolved in a buffered solution (e.g., a PBS solution) to the surface (101; e.g., a gold surface) of the working electrode (100) such that the capture molecule (102; e.g., capture antibody) adsorbs onto the surface (101; e.g., a gold surface) to generate an occupied capture surface. For example, the capture molecule (102; e.g., capture antibody) may spontaneously adsorb onto the electrode via the binding affinity of the capture molecule (102; e.g., capture antibody) to the electrode.

In some embodiments, the capture molecule (102; e.g., capture antibody) may be incubated on the surface (101; e.g., a gold surface) of the working electrode (100) in a humidified chamber for a period of time. In some embodiments, the capture molecule (102; e.g., capture antibody) is incubated in a humidified chamber overnight. In some embodiments, the capture molecule (102; e.g., capture antibody) is incubated in a humidified chamber for about 8 hours. In some embodiments, the capture molecule (102; e.g., capture antibody) is incubated in a humidified chamber for about 12 hours. In some embodiments, the capture molecule (102; e.g., capture antibody) is incubated in a humidified chamber for about 16 hours. In some embodiments, the capture molecule (102; e.g., capture antibody) is incubated in a humidified chamber for about 20 hours. In some embodiments, the capture molecule (102; e.g., capture antibody) is incubated in a humidified chamber for about 24 hours.

In some embodiments, the capture molecule (102; e.g., capture antibody) is incubated in a humidified chamber at about 4° C. In some embodiments, the capture molecule (102; e.g., capture antibody) is incubated in a humidified chamber at about 1° C. In some embodiments, the capture molecule (102; e.g., capture antibody) is incubated in a humidified chamber at about 10° C.

In certain embodiments, the capture molecule (102; e.g., capture antibody) may be incubated on the surface (101; e.g., a gold surface) of the working electrode (100) in a humidified chamber overnight at 4° C.

The buffered solution (e.g., a PBS solution) may help with the pH and keeping the surface (101; e.g., a gold surface) of the working electrode (100) moist during the coating step (i.e., during the application of the capture molecule (102; e.g., capture antibody).

Next, the method may comprise applying blocking molecules (105; e.g., bovine serum albumin molecules or Casein molecules) dissolved in a buffered solution (e.g., a blocking buffer; e.g., a blocking buffer comprising PBS) to the surface (101; e.g., a gold surface) of the working electrode (100) such that the blocking molecules (105; e.g., bovine serum albumin molecules) adsorb onto the surface (101; e.g., a gold surface) over the unoccupied capture surface. In other embodiments, the method comprises applying blocking molecules (105; e.g., bovine serum albumin molecules or Casein molecules) dissolved in a buffered solution (e.g., a blocking buffer; e.g., a blocking buffer comprising PBS) to at least one of the surfaces of the working electrode (100), the counter electrode (200), or the pseudo-reference electrode (300) such that the plurality of blocking molecules (105) adsorb onto the surface of at least one of the working electrode (100), counter electrode (200), or pseudo-reference electrode (300), over a remaining unoccupied capture surface.

In some embodiments, the method comprises applying blocking molecules (105; e.g., bovine serum albumin molecules or Casein molecules) dissolved in a buffered solution (e.g., a blocking buffer; e.g., a blocking buffer comprising PBS) to all sample contacting surfaces. Sample contacting surfaces may comprise electrode surfaces (e.g., the working electrode surface, the counter electrode surface, the reference electrode surface, or a combination thereof), non-electrode surfaces (e.g., non-conductive surface), or a combination thereof.

In some embodiments, the method may comprise removing the buffered solution containing the blocking molecules (105; e.g., bovine serum albumin molecules) from the surface (101; e.g., a gold surface) of the working electrode (100). In some embodiments, the method may comprise removing the buffered solution containing the blocking molecules (105; e.g., bovine serum albumin molecules) from the sample contacting surfaces.

The aforementioned method may further comprise washing the surface of the sensor (050), wherein the surface of the sensor comprises the working electrode surface, the counter electrode surface, the reference electrode surface, with a buffered detergent. In some embodiments, the surface of the sensor is washed with a buffered solution (e.g., PBST) and may be incubated for a period of time (e.g., about one to five minutes). The washing step may be repeated several times. In some embodiments, the washing step is repeated twice, for a total of three washes.

The method may further comprise adding a sample (e.g., a urine sample, a blood sample, or a saliva sample) to the surface of the sensor (050), e.g., the working electrode surface, the counter electrode surface, the reference electrode surface.

In some embodiments, a sample may be diluted in the buffered solution. In some embodiments, the final buffered solution is about 10% sample, volume per volume. For example, in some embodiments, the final buffered solution with a total volume of 200 mL may contain about 20 mL of sample (e.g., urine). In some embodiments, the final buffered solution is about 5% sample, or about 10%, or about 15%, or about 20%, volume per volume.

In some embodiments, the composition of the final buffered solution is specifically formulated based on at least one of the detection molecules (700; e.g., the detection antibody) or probe-enzyme conjugate (750). In some embodiments, the composition of the final buffered solution is specifically formulated based on at least one of the detection molecules (700; e.g., the detection antibody), or redox-capable enzyme (753), or redox reagent. In some embodiments, the composition of the final buffered solution comprises phosphate buffer saline (PBS), bovine serum albumin (BSA), casein, Polysorbate 20 (Tween 20), or a combination thereof. As a non-limiting example, in some embodiments, a buffered solution used to test a urine sample may contain 1% weight per volume bovine serum albumin (BSA) and/or 0.05% Polysorbate 20 (Tween 20) in a phosphate buffered saline solution with a pH of 7.4.

In some embodiments, the method further comprises using a vibration exciter to excite at least one of the capture molecules (102; e.g., capture antibodies), the blocking molecules (105; e.g., the bovine serum albumin molecules), the buffered solution, the buffered detergent, or a combination thereof.

In some embodiments, the method of fabricating a sensor, as described herein, comprises placing a volumetric cell (800) on the surface of a sensor (050).

The sensor (050) may comprise a working electrode (100), a counter electrode (200), and a pseudo-reference electrode (300). In some embodiments, the working electrode (100) includes a working electrode surface (101). In some embodiments, the method includes applying a capture molecule (102; e.g., capture antibody) dissolved in a buffered solution (e.g., a PBS solution) to the surface (101) of the working electrode (100) such that the capture molecule (102) adsorbs onto the surface (101) to generate an occupied capture surface.

The method may further comprise applying a plurality of blocking molecules (105) dissolved in a blocking solution to the surface (101) of the working electrode (100) such that the plurality of blocking molecules (105) adsorb onto the working electrode surface (101) over a remaining unoccupied capture surface. In some embodiments, the method includes applying blocking molecules (105) dissolved in a solution to at least one of the surfaces of the working electrode (100), the counter electrode (200), or the pseudo-reference electrode (300) such that the plurality of blocking molecules (105) adsorb onto the surface of at least one of the working electrode (100), counter electrode (200), or pseudo-reference electrode (300), over a remaining unoccupied capture surface. In some embodiments, the method includes removing the solution containing the plurality of blocking molecules (105) from the surface of the sensor (050). In some embodiments, the method includes washing the surface of the sensor (050).

In some embodiments, the method of fabricating a sensor further includes using a vibration exciter to excite at least one of the capture molecules (102), the plurality of blocking molecules (105), or the solution.

### Methods of Use

The present invention may further feature a method of using a Faradaic electrochemical based sensor (e.g., an Enzyme-Linked Immunosorbent Biosensor (ELIS-B) as described herein.

The method may comprise applying a sample (600), possibly containing a target biomarker (601), to the surface of a sensor (050). In some embodiments, the sample (600) is a urine sample. In some embodiments, the sample (600) is a blood sample. In some embodiments, the sample is a saliva sample.

As described above, in some embodiments, the sensor system may comprise a potentiostat (099), a working electrode (100) comprising a surface (101), and a capture molecule (102; e.g., a capture antibody) adsorbed onto the surface (101) to generate an occupied capture surface. The sensor system may further comprise blocking molecules (105; e.g., bovine serum albumin molecules or Casein molecules) disposed on the unoccupied captured surface.

In other embodiments, the sensor (050) comprises a capture antibody (102) adsorbed onto the capture surface of a working electrode (100). In some embodiments, a remaining unoccupied capture surface of the working electrode (100) is occupied by a plurality of bovine serum albumin molecules (105).

The method may further comprise applying a detection molecule (700; e.g., a detection antibody) to the surface of the sensor (050). In some embodiments, the detection molecule (700; the detection antibody) is biotinylated (e.g., bound to biotin). In some embodiments, the detection molecule (700; the detection antibody) is designed to bind to the target biomarker (601) to form a detection molecule-target biomarker complex (702). In some embodiments, the method includes incubating the solution comprising the sample (600) and the detection molecule (700) on the surface of the sensor (050) for a period of time, such that the target biomarker (601) possibly present in the sample (600) may bind the detection molecule (700; the detection antibody) to form the detection molecule-target biomarker complex (702).

In some embodiments, the solution comprising the sample (600), possibly containing a target biomarker (601), and the detection molecule (700) is incubated for about 15 to 20 minutes. In some embodiments, the solution comprising the sample (600) possibly containing a target biomarker (601) and the detection molecule (700) is incubated for about 5 minutes, or about 10 minutes, or about 15 minutes, or about 20 minutes, or about 25 minutes. In some embodiments, the solution comprising the sample (600) possibly containing a target biomarker (601) and the detection molecule (700) is incubated for about 30 minutes. In some embodiments, the solution comprising the sample (600) possibly containing a target biomarker (601) and the detection molecule (700) is incubated for about 60 minutes. In some embodiments, the solution comprising the sample (600) possibly containing a target biomarker (601) and the detection molecule (700) is incubated for over 60 minutes.

In some embodiments, the capture molecule (102; e.g., capture antibody) and the detection molecule-target biomarker complex (702; e.g., the detection antibody-target biomarker complex) form a sandwich complex (703) upon binding of the capture molecule (102; e.g., capture antibody) to the detection molecule-target biomarker complex (702; e.g., the detection antibody-target biomarker complex). The binding between the capture molecule and the complex may occur via interactions between the capture molecule (102; e.g, capture antibody) and a target biomarker portion of the complex (702). In some embodiments, the sandwich complex (703) includes the capture molecule (102; e.g, capture antibody) bound to the detection molecule-target biomarker complex (702). See FIG. 1.

In accordance with FIG. 1, in some embodiments, the method may also comprise applying a probe-enzyme conjugate (750) to the surface of the sensor (050). In some embodiments, the probe-enzyme conjugate (750) may comprise a probe (752; e.g., a streptavidin or avidin tetramer) bound to an enzyme (751). In some embodiments, the enzyme comprises a redox-capable enzyme. In some embodiments, the redox-capable enzyme is horseradish peroxidase. In some embodiments, the enzyme is horseradish peroxidase.

In some embodiments, the probe-enzyme conjugate (750) is designed to interact with the sandwich complex (703). In some embodiments, the redox-capable enzyme (e.g., 751, horseradish peroxidase) of the probe-enzyme conjugate (750) interacts with the biotinylated detection molecule (e.g., the biotinylated detection antibody) of the sandwich complex.

The method may further comprise applying a substrate solution comprising a chromogenic substrate or oxidative substrate (e.g., 3,3′,5,5′-tetramethylbenzidine (TMB), 2,2′-Azino-bis-(3-ethylbenzothiazoline-6-sulfonic acid) (ABTS), or 3,3′-Diaminobenzidine (DAB)) to the surface of the sensor. In certain embodiments, the method comprises applying a substrate solution comprising a phosphate-citrate buffer solution, hydrogen peroxide, and 3,3′,5,5′-tetramethylbenzidine (TMB) to the surface of the sensor (050).

Without wishing to limit the present invention to any theory or mechanism, it is believed, the redox-capable enzyme (e.g., horseradish peroxidase) catalyzes a redox reaction. For example, the TMB donates electrons to catalyze hydrogen peroxide into water. Thus, TMB is oxidized to form an oxidized form of TMB. In some embodiments, the oxidized form of TMB comprises an electrically-detectable compound (e.g., ferrocyanide). The electrically-detectable compound is reduced to an unoxidized form of TMB via a working electrode reducing potential applied to the working electrode (100). In some embodiments, the working electrode reduces potential transfer of electrons from the working electrode (100) to the electrically-detectable compound, thus reducing the electrically-detectable compound to the unoxidized form of TMB. In some embodiments, a possible working electrode-counter electrode current change is thus generated. In some embodiments, the working electrode-counter electrode current change is measured as a function of time by a potentiostat (099). In some embodiments, it is measured using chronoamperometry. In some embodiments, the working electrode-counter electrode current change is measured using differential pulse voltammetry. In some embodiments, the working electrode-counter electrode current change may be used to perform at least one of detection of the target biomarker (601) or quantification of the target biomarker (601).

Differential pulse voltammetry (DPV) scans from a non-reactive voltage to a reactive voltage for the studied reaction (e.g., redox of the substrate solution as described herein). The rate of change in the current will spike at the redox voltage/potential of the substrate. The height of the peak will be proportional to the concentration of the substrate solution. In some embodiments, samples described herein in which the target biomarker (601) is present will produce a large peak, whereas samples in which the target biomarker (601) is not present will produce a small peak. In some embodiments, samples described herein in which the target biomarker (601) is present will produce a large peak compared to a control sample. In some embodiments, the control sample does not contain the target biomarker. In some embodiments, samples in which the target biomarker (601) is not present will produce a small peak compared to a control sample (e.g., a control sample that contains the target biomarker).

In some embodiments, the method further includes using a vibration exciter to excite at least one of the capture antibody (102), the plurality of bovine serum albumin molecules (105), the sample (600), the target biomarker (601), the detection antibody (700), the detection antibody-target biomarker complex (702), the sandwich complex (703), the probe-enzyme conjugate (750), the probe (751), the enzyme, the phosphate-citrate buffer solution, the hydrogen peroxide, or the TMB.

In some embodiments, the method includes applying the sample (600) and the detection molecule (700; e.g., the detection antibody) to the surface of the sensor together, and then the probe-enzyme conjugate (750) is applied to the surface of the sensor (050) after. Alternatively, the method may comprise applying the detection molecule (700; e.g., a detection antibody) to the surface of the sensor (050) before the sample (600) is applied to the surface of the sensor (050), and the probe-enzyme conjugate (750) is applied to the surface of the sensor (050) after the sample (600) is applied. In some embodiments, this may improve the ability of the method to detect and/or quantify the target biomarker and/or target marker.

Alternatively, as shown in FIG. 9, the method may comprise applying a sample (600) possibly containing a target biomarker (601) and a detection molecule (700; e.g., a detection antibody) to the surface of the sensor (050), either simultaneously or separately. In some embodiments, the detection molecule (700; e.g., a detection antibody) is bound to a redox-capable enzyme (e.g., horseradish peroxidase).

In some embodiments, the detection molecule (700; the detection antibody) is designed to bind to the target biomarker (601) to form a detection molecule-target biomarker complex (702). For example, the redox-capable enzyme (e.g., horseradish peroxidase) may be bound to a portion of the first variable region, and the target biomarker (601) may bind to a position of the second variable region). In some embodiments, the method includes incubating the solution comprising the sample (600) and the detection molecule (700) on the surface of the sensor (050) for a period of time, such that the target biomarker (601) possibly present in the sample (600) may bind the detection molecule (700; the detection antibody) to form the detection molecule-target biomarker complex (702).

In some embodiments, the solution comprising the sample (600) possibly containing a target biomarker (601) and the detection molecule (700) is incubated for about 15 to 20 minutes. In some embodiments, the solution comprising the sample (600) possibly containing a target biomarker (601) and the detection molecule (700) is incubated for about 5 minutes, or about 10 minutes, or about 15 minutes, or about 20 minutes, or about 25 minutes. In some embodiments, the solution comprising the sample (600) possibly containing a target biomarker (601) and the detection molecule (700) is incubated for about 30 minutes. In some embodiments, the solution comprising the sample (600) possibly containing a target biomarker (601) and the detection molecule (700) is incubated for about 60 minutes. In some embodiments, the solution comprising the sample (600) possibly containing a target biomarker (601) and the detection molecule (700) is incubated for over 60 minutes.

In some embodiments, the capture molecule (102; e.g., capture antibody) and the detection molecule-target biomarker complex (702; e.g., the detection antibody-target biomarker complex) form a sandwich complex (703) upon binding of the capture molecule (102; e.g., capture antibody) to the detection molecule-target biomarker complex (702; e.g., the detection antibody-target biomarker complex). The binding between the capture molecule and the complex may occur via interactions between the capture molecule (102; e.g, capture antibody) and a target biomarker portion of the complex (702). In some embodiments, the sandwich complex (703) includes the capture molecule (102; e.g, capture antibody) bound to the detection molecule-target biomarker complex (702). See FIG. 9.

The method may further comprise applying a substrate solution comprising a chromogenic substrate or oxidative substrate (e.g., 3,3′,5,5′-tetramethylbenzidine (TMB), 2,2′-Azino-bis-(3-ethylbenzothiazoline-6-sulfonic acid) (ABTS), or 3,3′-Diaminobenzidine (DAB)) to the surface of the sensor. In certain embodiments, the method comprises applying a substrate solution comprising a phosphate-citrate buffer solution, hydrogen peroxide, and 3,3′,5,5′-tetramethylbenzidine (TMB) to the surface of the sensor (050).

The present invention features a method of using a sensor system to analyze a sample. In some embodiments, the method includes applying a sample (600) possibly containing a target marker (601) to the surface of a sensor (050). In some embodiments, the sensor (050) includes a capture molecule (102) adsorbed onto an occupied capture surface of an electrode surface (101) of a working electrode (100). In some embodiments, the remaining unoccupied capture surface of the working electrode (100) is occupied by a plurality of blocking molecules (105). In some embodiments, the method includes applying a detection molecule (700) to the surface of the sensor (050). In some embodiments, the detection molecule (700) is designed to bind to the target marker (601) to form a detection molecule-target marker complex (702). In some embodiments, the method includes incubating the sample (600) on the sensor (050) for a period of time, such that the target marker (601) possibly present in the sample (600) may bind the detection molecule (700) to form the detection molecule-target marker complex (702). In some embodiments, the method includes wherein the capture molecule (102) and the detection molecule-target marker complex (702) form a sandwich complex (703) upon binding of the capture molecule (102) to the detection molecule-target marker complex (702). In some embodiments, the sandwich complex (703) includes the capture molecule (102) bound to the detection molecule-target marker complex (702). In some embodiments, the method includes applying a redox-capable enzyme (753) to the surface of the sensor (050). In some embodiments, the method includes applying a redox reagent to the surface of the sensor (050). In some embodiments, the redox-capable enzyme (753) performs at least one of electron donation or electron acceptance with the redox reagent. In some embodiments, the oxidation state of the redox reagent is changed to produce an oxidized or reduced redox reagent. In some embodiments, the oxidized or reduced redox reagent comprises an electrically-detectable compound. In some embodiments, the electrically-detectable compound is oxidized or reduced via a working electrode potential applied to the working electrode (100). In some embodiments, the working electrode potential transfers electrons to or from the working electrode (100) to or from the electrically-detectable compound, thus oxidizing or reducing the electrically-detectable compound to its original unoxidized or unreduced state. In some embodiments, a possible change in at least one of current or voltage in or between at least one of the working electrode (100), a counter electrode (200), or a pseudo-reference electrode (300) is thus generated. In some embodiments, the possible change in at least one of current or voltage in or between at least one of the working electrode (100), counter electrode (200), or pseudo-reference electrode (300) is measured by an electrode analysis device (099). In some embodiments, the possible change in at least one of current or voltage in or between at least one of the working electrode (100), counter electrode (200), or pseudo-reference electrode (300) may be used to perform at least one of detection of the target marker (601) or quantification of the target marker (601).

In some embodiments, the method includes using a vibration exciter to excite at least one of the capture molecules (102), the plurality of blocking molecules (105), the sample (600), the target marker (601), the detection molecule (700), the detection molecule-target marker complex (702), the sandwich complex (703), the redox-capable enzyme (753), or the redox reagent.

In some embodiments, the method includes wherein the working electrode surface (101) comprises carbon. In some embodiments, the method includes wherein the working electrode surface (101) comprises gold.

In some embodiments, the method includes wherein the detection molecule (700) is applied to the surface of the sensor (050) before the sample (600) is applied to the surface of the sensor (050), and wherein the redox-capable enzyme (753) is applied to the surface of the sensor (050) after the sample (600) is applied.

### EMBODIMENTS

The following embodiments are intended to be illustrative only and not to be limiting in any way.

Embodiment 1: A sensor system (e.g., a Faradaic electrochemical based sensor system) for analysis of a sample, said sensor system comprising: a) an electrode analysis device (099; e.g., a potentiostat), b) a working electrode (100) comprising a surface (101) and a capture molecule (102) adsorbed onto the working electrode surface to generate an occupied capture surface, a remaining unoccupied capture surface occupied by a plurality of blocking molecules (105); wherein the surface (101) of the working electrode (100) is kept moist; c) a counter electrode (200); and d) a pseudo-reference electrode (300), wherein said working electrode (100), said counter electrode (200), said pseudo-reference electrode (300), and said electrode analysis device (099) are electrically connected, wherein said electrode analysis device (099; e.g., a potentiostat) analyzes at least one parameter selected from current or voltage in or between at least one of the working electrode (100), counter electrode (200), or pseudo-reference electrode (300).

Embodiment 2: An electrochemical-based sensor system for analysis of a sample, the sensor system comprising: a) a potentiostat (099); b) a working electrode (100) comprising a surface (101) and a capture molecule (102) adsorbed onto a proportion of the surface (101) to generate an occupied capture surface; wherein the surface (101) of the working electrode (100) is kept moist; c) a counter electrode; and d) a pseudo-reference electrode; wherein the working electrode (100), the counter electrode (200), the pseudo-reference electrode (300), and the potentiostat (099) are electrically connected, wherein the potentiostat (099) controls a voltage difference between the working electrode (100) and the pseudo-reference electrode (300) by injecting current through the counter electrode (200), wherein said potentiostat (099) measures a working electrode-counter electrode current change at the working electrode (100).

Embodiment 3: The sensor system of embodiment 1 or embodiment 2, wherein the working electrode (100) is a gold electrode comprising a gold surface (101).

Embodiment 4: The sensor system of embodiment 3, wherein the counter electrode (200) is a gold electrode.

Embodiment 5: The sensor system of embodiment 1 or embodiment 2, wherein the working electrode (100) is a carbon electrode comprising a carbon surface (101).

Embodiment 6: The sensor system of embodiment 5, wherein the counter electrode (200) is a carbon electrode.

Embodiment 7: The sensor system of any one of embodiments 1-6, wherein the pseudo-reference electrode (300) is a Silver/Silver Chloride (Ag/AgCl) electrode.

Embodiment 8: The sensor system of any one of embodiments 1-7, further comprising a substrate; wherein the working electrode, the counter electrode, and the reference electrode are ablated or plated on top of the substrate.

Embodiment 9: The sensor system of embodiment 8, wherein the substrate is made of polyethylene terephthalate (PET).

Embodiment 10: The sensor system of any one of embodiments 1-9, further comprising an insulating dielectric layer; wherein the insulating dielectric layer is placed on at least a portion of the working electrode surface, a counter electrode surface, and a reference electrode surface not intended to be exposed to a sample.

Embodiment 11: The sensor system of any one of embodiments 1-10, wherein the capture molecule (102) is a capture antibody.

Embodiment 12: The sensor system of any one of embodiments 1-11, wherein blocking molecules (105) are disposed on an unoccupied capture surface of the working electrode (100).

Embodiment 13: The sensor system of embodiment 12, wherein the blocking molecules (105) are bovine serum albumin molecules, casein, or a combination thereof.

Embodiment 14: A method of analyzing a sample using a sensor system according to any one of embodiments 1-13, the method comprising: adding a sample to a surface of the sensor.

Embodiment 15: The method of embodiment 14, wherein the sample is a urine sample, a blood sample, or a saliva sample.

Embodiment 16: The method of embodiment 14 or embodiment 15, wherein the sample potentially comprises a target biomarker (601).

Embodiment 17: The method of any one of embodiments 14-16, wherein the sample is diluted in a buffered solution.

Embodiment 18: The method of embodiment 17, wherein the buffered solution comprises a detection molecule (700) and a probe-enzyme conjugate (750) comprising a probe (751) bound to an enzyme.

Embodiment 19: The method of embodiment 18, wherein the detection molecule (700) is a detection antibody.

Embodiment 20: The method of embodiment 18 or embodiment 19, detection molecule (700) is biotinylated.

Embodiment 21: The method of any one of embodiments 18-20, wherein the detection molecule (700) is designed to bind to the target biomarker (601) to form a detection molecule-target biomarker complex (702), wherein the capture molecule (102) and the detection molecule-target biomarker complex (702) form a sandwich complex (703) upon binding of the capture molecule (102) to the detection molecule-target biomarker complex (702) via interactions between the capture molecule (102) and a target biomarker portion of the detection molecule-target biomarker complex (702), said sandwich complex (703) comprising the capture molecule (102) bound to the detection molecule-target biomarker complex (702).

Embodiment 22: The method of any one of embodiments 18-21, wherein the probe is a streptavidin tetramer or an avidin tetramer.

Embodiment 23: The method of any one of embodiments 18-22, wherein the probe is bound to a redox-capable enzyme.

Embodiment 24: The method of embodiment 23, wherein the enzyme is horseradish peroxidase.

Embodiment 25: The method of any one of embodiments 21-24, wherein the probe-enzyme conjugate (750) is designed to interact with the sandwich complex (703) wherein the probe (e.g., the streptavidin tetramer or the avidin tetramer) interacts with the biotinylated detection molecule (700) of the sandwich complex (703).

Embodiment 26: The method of embodiment 17, wherein the buffered solution comprises a detection molecule (700) bound to an enzyme.

Embodiment 27: he method of embodiment 26, wherein the detection molecule (700) is a detection antibody.

Embodiment 28: The method of embodiment 26 or embodiment 27, wherein the detection molecule (700) is designed to bind to the target biomarker (601) to form a detection molecule-target biomarker complex (702), wherein the capture molecule (102) and the detection molecule-target biomarker complex (702) form a sandwich complex (703) upon binding of the capture molecule (102) to the detection molecule-target biomarker complex (702) via interactions between the capture molecule (102) and a target biomarker portion of the detection molecule-target biomarker complex (702), said sandwich complex (703) comprising the capture molecule (102) bound to the detection molecule-target biomarker complex (702).

Embodiment 29: The method of any one of embodiments 26-28, wherein the probe is bound to a redox-capable enzyme; wherein the redox-capable enzyme is horseradish peroxidase.

Embodiment 30: The method of any one of embodiments 14-29, further comprising adding a substrate solution to the surface of the sensor.

Embodiment 31: The method of embodiment 30, wherein the substrate solution comprises a phosphate-citrate buffer solution, hydrogen peroxide, and a chromogenic substrate. wherein the chromogenic substrate is selected from a group comprising 3,3′,5,5′-tetramethylbenzidine (TMB), 2,2′-Azino-bis-(3-ethylbenzothiazoline-6-sulfonic acid) (ABTS), or 3,3′-Diaminobenzidine (DAB).

Embodiment 32: The method of any one of embodiments 14-31, wherein the redox-capable enzyme (e.g.,horseradish peroxidase) catalyzes a redox reaction, wherein the chromogenic substrate (e.g., TMB) donates electrons to catalyze hydrogen peroxide into water, thus oxidizing the chromogenic substrate (e.g., TMB) into an oxidized form of the chromogenic substrate (e.g., TMB), wherein the oxidized form of the chromogenic substrate (e.g., TMB) comprises an electrically-detectable compound, wherein the electrically-detectable compound is reduced to an unoxidized form of the chromogenic substrate (e.g., TMB) via a working electrode reducing potential applied to the working electrode (100), said working electrode reducing potential transferring electrons from the working electrode (100) to the electrically-detectable compound, thus reducing the electrically-detectable compound to the unoxidized form of the chromogenic substrate (e.g., TMB), wherein the possible working electrode-counter electrode current change is thus generated, wherein said working electrode-counter electrode current change is measured as a function of time by the potentiostat using chronoamperometry, and wherein said working electrode-counter electrode current change may be used to perform at least one of detection of the target biomarker (601) or quantification of the target biomarker (601).

Embodiment 33: The method of any one of embodiments 14-32, further comprising vibrating the sample with a vibration exciter to excite at least one of the capture molecule (102), the blocking molecules (105), the sample (600), the target biomarker (601), the detection molecule (700), the detection molecule-target biomarker complex (702), the sandwich complex (703), the probe-enzyme conjugate (750), the probe (751), the enzyme, the phosphate-citrate buffer solution, the hydrogen peroxide, or the chromogenic substrate (e.g., TMB).

Embodiment 34: The method of any one of embodiments 14-17, wherein the buffered solution comprises a detection molecule (700), a redox-capable enzyme (753); and a redox reagent.

Embodiment 35: The method of embodiment 34, wherein the redox-capable enzyme (753) performs at least one of electron donation or electron acceptance with the redox reagent, wherein the oxidation state of the redox reagent is changed to produce an oxidized or reduced redox reagent, wherein the oxidized or reduced redox reagent comprises an electrically-detectable compound, wherein the electrically-detectable compound is oxidized or reduced via a working electrode potential applied to the working electrode (100), said working electrode potential transferring electrons to or from the working electrode (100) to or from the electrically-detectable compound, thus oxidizing or reducing the electrically-detectable compound to its original unoxidized or unreduced state, wherein a possible change in at least one of current or voltage in or between at least one of the working electrode (100), counter electrode (200), or pseudo-reference electrode (300) is thus generated, wherein said possible change in at least one of current or voltage in or between at least one of the working electrode (100), counter electrode (200), or pseudo-reference electrode (300) is measured by the electrode analysis device (099), and wherein said potential possible change in at least one of current or voltage in or between at least one of the working electrode (100), counter electrode (200), or pseudo-reference electrode (300) may be used to perform at least one of detection of the target marker (601) or quantification of the target marker (601).

Embodiment 36: A sensor system for analysis of a sample, said sensor system comprising: a) a potentiostat (099); b) a working electrode (100) comprising a gold surface (101) and a capture antibody (102) adsorbed onto the gold surface (101) over an occupied capture surface, a remaining unoccupied capture surface occupied by a plurality of bovine serum albumin molecules (105); c) a gold counter electrode (200); d) an Ag/AgCl pseudo-reference electrode (300), wherein said working electrode (100), said gold counter electrode (200), said Ag/AgCl pseudo-reference electrode (300), and said potentiostat (099) are electrically connected, wherein said potentiostat (099) controls a voltage difference between the working electrode (100) and the Ag/AgCl pseudo-reference electrode (300) by injecting current through the gold counter electrode (200), wherein said potentiostat (099) measures a working electrode-counter electrode current change at the working electrode (100); e) a substrate comprising PET (polyethylene terephthalate); and f) an insulating dielectric.

Embodiment 37: The sensor system of embodiment 36, further comprising a) a sample (600) possibly containing a target biomarker (601); b) a detection antibody (700), wherein the detection antibody (700) is biotinylated, said detection antibody (700) designed to bind to the target biomarker (601) to form a detection antibody-target biomarker complex (702), wherein the capture antibody (102) and the detection antibody-target biomarker complex (702) form a sandwich complex (703) upon binding of the capture antibody (102) to the detection antibody-target biomarker complex (702) via interactions between the capture antibody (102) and a target biomarker portion of the detection antibody-target biomarker complex (702), said sandwich complex (703) comprising the capture antibody (102) bound to the detection antibody-target biomarker complex (702); c) a probe-enzyme conjugate (750) comprising a probe (e.g., a streptavidin tetramer or an avidin tetramer) bound to an enzyme (e.g., a redox capable enzyme, e.g., horseradish peroxidase); said probe-enzyme conjugate (750) designed to interact with the sandwich complex (703) wherein the probe (e.g., a streptavidin tetramer or an avidin tetramer) of the probe-enzyme conjugate (750) interacts with the biotinylated detection antibody (700) of the sandwich complex (703); and a substrate solution comprising a phosphate-citrate buffer solution, hydrogen peroxide, and 3,3′,5,5′-tetramethylbenzidine (TMB); wherein the horseradish peroxidase catalyzes a redox reaction wherein TMB donates electrons to catalyze hydrogen peroxide into water; wherein TMB is thus oxidized to form an oxidized form of TMB, wherein the oxidized form of TMB comprises an electrically-detectable compound, wherein the electrically-detectable compound is reduced to an unoxidized form of TMB via a working electrode reducing potential applied to the working electrode (100), said working electrode reducing potential transferring electrons from the working electrode (100) to the electrically-detectable compound, thus reducing the electrically-detectable compound to the unoxidized form of TMB, wherein the possible working electrode-counter electrode current change is thus generated, wherein said working electrode-counter electrode current change is measured as a function of time by the potentiostat using chronoamperometry, and wherein said working electrode-counter electrode current change may be used to perform at least one of detection of the target biomarker (601) or quantification of the target biomarker (601).

Embodiment 38: The sensor system of embodiment 36 or embodiment 37, wherein the system further comprises a vibration exciter to excite at least one of the capture antibodies (102), the plurality of bovine serum albumin molecules (105), the sample (600), the target biomarker (601), the detection antibody (700), the detection antibody-target biomarker complex (702), the sandwich complex (703), the probe-enzyme conjugate (750), the probe (751), the enzyme, the phosphate-citrate buffer solution, the hydrogen peroxide, or the TMB.

Embodiment 39: A sensor system for analysis of a sample, said sensor system comprising: a) an electrode analysis device (099); b) a working electrode (100) comprising a working electrode surface (101) and a capture molecule (102) adsorbed onto the working electrode surface over an occupied capture surface, a remaining unoccupied capture surface occupied by a plurality of blocking molecules (105); c) a counter electrode (200); and d) a pseudo-reference electrode (300), wherein said working electrode (100), said counter electrode (200), said pseudo-reference electrode (300), and said electrode analysis device (099) are electrically connected, wherein said electrode analysis device (099) analyzes at least one parameter selected from current or voltage in or between at least one of the working electrode (100), counter electrode (200), or pseudo-reference electrode (300).

Embodiment 40: The sensor system of embodiment 37, further comprising a a) sample (600) possibly containing a target marker (601); b) a detection molecule (700), said detection molecule (700) designed to bind the target biomarker (601) to form a detection molecule-target biomarker complex (702), wherein the capture molecule (102) and the detection molecule-target biomarker complex (702) form a sandwich complex (703) upon binding of the capture molecule (102) to the detection molecule-target biomarker complex (702), said sandwich complex (703) comprising the capture molecule (102) bound to the detection molecule-target marker complex (702); c) a redox-capable enzyme (753); and d) a redox reagent; wherein the redox-capable enzyme (753) performs at least one of electron donation or electron acceptance with the redox reagent, wherein the oxidation state of the redox reagent is changed to produce an oxidized or reduced redox reagent, wherein the oxidized or reduced redox reagent comprises an electrically-detectable compound, wherein the electrically-detectable compound is oxidized or reduced via a working electrode potential applied to the working electrode (100), said working electrode potential transferring electrons to or from the working electrode (100) to or from the electrically-detectable compound, thus oxidizing or reducing the electrically-detectable compound to its original unoxidized or unreduced state, wherein a possible change in at least one of current or voltage in or between at least one of the working electrode (100), counter electrode (200), or pseudo-reference electrode (300) is thus generated, wherein said possible change in at least one of current or voltage in or between at least one of the working electrode (100), counter electrode (200), or pseudo-reference electrode (300) is measured by the electrode analysis device (099), and wherein said potential possible change in at least one of current or voltage in or between at least one of the working electrode (100), counter electrode (200), or pseudo-reference electrode (300) may be used to perform at least one of detection of the target marker (601) or quantification of the target marker (601).

Embodiment 41: A method of fabricating a sensor (e.g., a Faradaic electrochemical based sensor system) comprising: a) applying a capture molecule (102) dissolved in a buffered solution to a surface (101) of a working electrode (100); such that the capture molecule (102) adsorbs onto the surface (101) to generate an occupied capture surface; wherein hydration is maintained such that the surface (100) of the working electrode (100) is moist; applying blocking molecules (105) dissolved in a buffered solution to a surface of a sensor; wherein the sensor comprises the working electrode, a counter electrode, and a reference electrode, wherein the surface of the sensor comprises all sample contacting surfaces comprising all electrode surfaces, non-electrode surfaces or a combination thereof, wherein the blocking molecules (105) adsorb onto at least one of the electrode surfaces over a remaining unoccupied capture surface; and removing the buffered solution containing the blocking molecules (105) from the surface of the sensor (050).

Embodiment 42: A method of fabricating a sensor (e.g., a Faradaic electrochemical based sensor system) comprising: a) applying a capture molecule (102) dissolved in a buffered solution to a surface (101) of a working electrode (100); such that the capture molecule(102) adsorbs onto the surface (101) to generate an occupied capture surface; wherein hydration is maintained such that the surface (100) of the working electrode (100) is moist; applying blocking molecules (105) dissolved in a buffered solution to a surface of a sensor; wherein the sensor is according to any one of embodiments 1-13, wherein the surface of the sensor comprises all sample contacting surfaces comprising all electrode surfaces, non-electrode surfaces or a combination thereof, wherein the blocking molecules (105) adsorb onto at least one of the electrode surfaces over a remaining unoccupied capture surface; and removing the buffered solution containing the blocking molecules (105) from the surface of the sensor (050).

Embodiment 43: The method of embodiment 41 or embodiment 42, further comprising placing a volumetric cell (800) on the surface of the sensor (050).

Embodiment 44: The method of any one of embodiments 41-43, further comprising washing the surface of the sensor (050) with a buffered detergent.

Embodiment 45: The method of any one of embodiments 41-44, further comprising using a vibration exciter to excite at least one of the capture molecule/capture antibody (102), the blocking molecules (105), the buffered solution, or the buffered detergent.

Embodiment 46: A method of using a sensor system to analyze a sample, the method comprising: a) applying a sample (600) possibly containing a target biomarker (601) to a surface of a sensor (050), wherein the sensor (050) comprises a capture molecule (102) adsorbed onto an occupied capture surface of a surface (101) of a working electrode (100), a remaining unoccupied capture surface of the working electrode (100) being occupied by a plurality of blocking molecules (105); b) applying a detection molecule (700) to the surface of the sensor (050); wherein the detection molecule is biotinylated; wherein the detection molecule (700) designed to bind to the target biomarker (601) to form a detection molecule-target biomarker complex (702); c) incubating the sample (600) and the detection antibody (700) on the surface of the sensor (050) for a period of time, such that the target biomarker (601) binds the detection molecule (700) to form the detection molecule-target biomarker complex (702); wherein the capture molecule (102) and the detection molecule-target biomarker complex (702) form a sandwich complex (703) upon binding of the capture molecule (102) to the detection molecule-target biomarker complex (702) via interactions between the capture molecule (102) and a target biomarker portion of the detection molecule-target biomarker complex (702), said sandwich complex (703) comprising the capture molecule (102) bound to the detection molecule-target biomarker complex (702); d) applying a probe-enzyme conjugate (750) to the surface of the sensor (050), wherein the probe-enzyme conjugate (750) comprising a streptavidin or avidin tetramer bound to a redox-capable enzyme; wherein the streptavidin or avidin tetramer of the probe-enzyme conjugate (750) interacts with the biotinylated detection molecule (700) of the sandwich complex; and e) applying a substrate solution comprising a chromogenic substrate; wherein the redox-capable enzyme catalyzes a redox reaction in which the chromogenic substrate donates electrons to catalyze hydrogen peroxide into water; wherein the chromogenic substrate is thus oxidized to form an oxidized form of the chromogenic substrate; wherein the oxidized form of the chromogenic substrate comprises an electrically-detectable compound, wherein the electrically-detectable compound is reduced to an unoxidized form of the chromogenic substrate via a working electrode reducing potential applied to the working electrode (100), said working electrode reducing potential transferring electrons from the working electrode (100) to the electrically-detectable compound, thus reducing the electrically-detectable compound to the unoxidized form of the chromogenic substrate, wherein a possible working electrode-counter electrode current change is thus generated, wherein said working electrode-counter electrode current change is measured as a function of time by a potentiostat (099) using chronoamperometry, and wherein said working electrode-counter electrode current change may be used to perform at least one of detection of the target biomarker (601) or quantification of the target biomarker (601).

Embodiment 47: A method of using a sensor system to analyze a sample, the method comprising: a) applying a sample (600) possibly containing a target biomarker (601) to a surface of a sensor (050), wherein the sensor (050) comprises a capture molecule (102) adsorbed onto an occupied capture surface of a surface (101) of a working electrode (100), a remaining unoccupied capture surface of the working electrode (100) being occupied by a plurality of blocking molecules (105); b) applying a detection molecule (700) to the surface of the sensor (050); wherein the detection molecule is bound to a redox-capable enzyme; wherein the detection molecule (700) designed to bind to the target biomarker (601) to form a detection molecule-target biomarker complex (702); c) incubating the sample (600) and the detection molecule (700) on the surface of the sensor (050) for a period of time, such that the target biomarker (601) binds the detection molecule (700) to form the detection molecule-target biomarker complex (702); wherein the capture molecule (102) and the detection molecule-target biomarker complex (702) form a sandwich complex (703) upon binding of the capture molecule (102) to the detection molecule-target biomarker complex (702) via interactions between the capture molecule (102) and a target biomarker portion of the detection molecule-target biomarker complex (702), said sandwich complex (703) comprising the capture molecule (102) bound to the detection molecule-target biomarker complex (702); and d) applying a substrate solution comprising a chromogenic substrate; wherein the redox-capable enzyme catalyzes a redox reaction in which the chromogenic substrate donates electrons to catalyze hydrogen peroxide into water; wherein the chromogenic substrate is thus oxidized to form an oxidized form of the chromogenic substrate; wherein the oxidized form of the chromogenic substrate comprises an electrically-detectable compound, wherein the electrically-detectable compound is reduced to an unoxidized form of the chromogenic substrate via a working electrode reducing potential applied to the working electrode (100), said working electrode reducing potential transferring electrons from the working electrode (100) to the electrically-detectable compound, thus reducing the electrically-detectable compound to the unoxidized form of the chromogenic substrate, wherein a possible working electrode-counter electrode current change is thus generated, wherein said working electrode-counter electrode current change is measured as a function of time by a potentiostat (099) using chronoamperometry, and wherein said working electrode-counter electrode current change may be used to perform at least one of detection of the target biomarker (601) or quantification of the target biomarker (601).

Embodiment 48: The method of embodiment 46 or embodiment 47, further comprising using a vibration exciter to excite at least one of the capture molecules (102), the plurality of blocking molecules (105), the sample (600), the target biomarker (601), the detection molecule (700), the detection molecule-target biomarker complex (702), the sandwich complex (703), the probe-enzyme conjugate (750), the probe (751), the enzyme, the phosphate-citrate buffer solution, the hydrogen peroxide, or the chromogenic substrate.

Embodiment 49: The method of any one of embodiments 46-48, wherein the detection antibody (700) is applied to the surface of the sensor (050) before the sample (600) is applied to the surface of the sensor (050), and wherein the probe-enzyme conjugate (750) is applied to the surface of the sensor (050) after the sample (600) is applied.

Embodiment 50: A method of using a sensor system to analyze a sample, the method comprising: a) applying a sample (600) possibly containing a target marker (601) to a surface of a sensor (050) fabricated according to the method of any one of embodiments 41-45, wherein the sensor (050) thus comprises capture molecules (102) adsorbed onto an occupied capture surface of the surface (101) of a working electrode (100), a remaining unoccupied capture surface of the working electrode (100) being occupied by a plurality of blocking molecules (105); b) applying a detection molecule (700) to the surface of the sensor (050), said detection molecule (700) designed to bind to the target marker (601) to form a detection molecule-target marker complex (702); c) incubating the sample (600) on the sensor (050) for a period of time, such that the target marker (601) possibly present in the sample (600) may bind the detection molecule (700) to form the detection molecule-target marker complex (702); wherein the capture molecule (102) and the detection molecule-target marker complex (702) form a sandwich complex (703) upon binding of the capture molecule (102) to the detection molecule-target marker complex (702), said sandwich complex (703) comprising the capture molecule (102) bound to the detection molecule-target marker complex (702); d) applying a redox-capable enzyme (753) to the surface of the sensor (050); and e) applying a redox reagent to the surface of the sensor (050); wherein the redox-capable enzyme (753) performs at least one of electron donation or electron acceptance with the redox reagent, wherein the oxidation state of the redox reagent is changed to produce an oxidized or reduced redox reagent, wherein the oxidized or reduced redox reagent comprises an electrically-detectable compound, wherein the electrically-detectable compound is oxidized or reduced via a working electrode potential applied to the working electrode (100), said working electrode potential transferring electrons to or from the working electrode (100) to or from the electrically-detectable compound, thus oxidizing or reducing the electrically-detectable compound to its original unoxidized or unreduced state, wherein a possible change in at least one of current or voltage in or between at least one of the working electrode (100), a counter electrode (200), or a pseudo-reference electrode (300) is thus generated, wherein said possible change in at least one of current or voltage in or between at least one of the working electrode (100), counter electrode (200), or pseudo-reference electrode (300) is measured by an electrode analysis device (099), and wherein said potential possible change in at least one of current or voltage in or between at least one of the working electrode (100), counter electrode (200), or pseudo-reference electrode (300) may be used to perform at least one of detection of the target marker (601) or quantification of the target marker (601).

Embodiment 51: The method of embodiment 50, further comprising using a vibration exciter to excite at least one of the capture molecule (102), the plurality of blocking molecules (105), the sample (600), the target marker (601), the detection molecule (700), the detection molecule-target marker complex (702), the sandwich complex (703), the redox-capable enzyme (753), or the redox reagent.

Embodiment 52: The method of embodiment 50 or embodiment 51, wherein the detection molecule (700) is applied to the surface of the sensor (050) before the sample (600) is applied to the surface of the sensor (050), and wherein the redox-capable enzyme (753) is applied to the surface of the sensor (050) after the sample (600) is applied.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

Reference numbers recited herein, in the drawings, and in the claims are solely for ease of examination of this patent application and are exemplary. The reference numbers are not intended in any way to limit the scope of the claims to the particular features having the corresponding reference numbers in the drawings.

What is claimed is:

1. A method of analyzing a sample using an electrochemical based sensor system, the method comprising:
a) applying a sample (600) possibly containing a target biomarker (601) to a surface of an electrochemical based sensor (050), wherein the electrochemical based sensor comprises:
i. an electrode analysis device (099);
ii. a working electrode (100) comprising a surface (101), wherein capture molecules (102) are adsorbed onto the surface (101) of the working electrode (100) to generate an occupied capture surface and a plurality of blocking molecules (105) are adsorbed onto a remaining unoccupied capture surface;
iii. a counter electrode (200);
iv. a pseudo-reference electrode (300), wherein the working electrode (100), the counter electrode (200), the pseudo-reference electrode (300), and the electrode analysis device (099) are electrically connected;
b) applying a detection molecule (700) and a redox-capable enzyme (753) to the surface of the sensor (050), wherein the detection molecule (700) is designed to bind to the target biomarker (601) to form a detection molecule-target biomarker complex (702);
c) incubating the sample (600), the detection molecule (700) and the redox-capable enzyme (753) on the sensor (050) for a period of time, such that the target biomarker (601) possibly present in the sample (600) may bind the detection molecule (700) to form the detection molecule-target biomarker complex (702);
wherein the capture molecule (102) and the detection molecule-target biomarker complex (702) form a sandwich complex (703) upon binding of the capture molecule (102) to the detection molecule-target biomarker complex (702), said sandwich complex (703) comprising the capture molecule (102) bound to the detection molecule-target biomarker complex (702); and
d) applying a substrate solution comprising a chromogenic substrate the surface of the sensor (050);
wherein the redox-capable enzyme catalyzes a redox reaction in which the chromogenic substrate donates electrons to catalyze hydrogen peroxide into water; wherein the chromogenic substrate is thus oxidized to form an oxidized form of the chromogenic substrate; wherein the oxidized form of the chromogenic substrate comprises an electrically-detectable compound, wherein the electrically-detectable compound is reduced to an unoxidized form of the chromogenic substrate via a working electrode reducing potential applied to the working electrode (100), said working electrode reducing potential transfers electrons from the working electrode (100) to the electrically-detectable compound, thus reducing the electrically-detectable compound to the unoxidized form of the chromogenic substrate, wherein a possible working electrode-counter electrode current change is thus generated, wherein said working electrode-counter electrode current change is measured as a function of time by a potentiostat (099) using chronoamperometry, and wherein said working electrode-counter electrode current change may be used to perform at least one of detection of the target biomarker (601) or quantification of the target biomarker (601).

2. The method of claim 1, wherein an electrode analysis device (099) comprises a potentiostat.

3. The method of claim 1, wherein the working electrode (100) is a gold electrode comprising a gold surface (101).

4. The method of claim 3, wherein the counter electrode (200) is a gold electrode.

5. The method of claim 1, wherein the pseudo-reference electrode (300) is a Silver/Silver Chloride (Ag/AgCl) electrode.

6. The method of claim 1, wherein the capture molecule (102) is a capture antibody.

7. The method of claim 1, wherein the plurality of blocking molecules (105) comprise bovine serum albumin molecules, casein molecules, or a combination thereof.

8. The method of claim 1, wherein the detection molecule (700) is a detection antibody.

9. The method of claim 1, wherein the redox-capable enzyme (753) is bound to the detection molecule (700).

10. The method of claim 1, wherein the detection molecule (700) is biotinylated and the redox-capable enzyme is bound to a probe comprising a streptavidin or avidin tetramer and forms a probe-enzyme conjugate (750).

11. The method of claim 10, wherein the probe-enzyme conjugate (750) is applied to the surface of the sensor (050) before step d), wherein the streptavidin or avidin tetramer of the probe-enzyme conjugate (750) interacts with the biotinylated detection molecule (700) of the sandwich complex.

12. The method of claim 1, wherein the detection molecule (700) is applied to the surface of the sensor (050) before the sample (600) is applied to the surface of the sensor (050).

13. The method of claim 1, wherein the redox-capable enzyme is horseradish peroxidase.

14. The method of claim 1, wherein the chromogenic substrate comprises 3,3′,5,5′-tetramethylbenzidine (TMB), 2,2′-Azino-bis-(3-ethylbenzothiazoline-6-sulfonic acid) (ABTS), or 3,3′-Diaminobenzidine (DAB).

15. The method of claim 1, wherein the substrate solution further comprises phosphate-citrate buffer solution and hydrogen peroxide.

16. The method of claim 1, wherein the sample is a urine sample, a blood sample, or a saliva sample.

17. The method of claim 1, further comprising vibrating the sample with a vibration exciter to excite at least one of the capture molecule (102), the plurality of blocking molecules (105), the sample (600), the target biomarker (601), the detection molecule (700), the detection molecule-target biomarker complex (702), the sandwich complex (703), the redox-capable enzyme, or the chromogenic substrate.
